SEQUENCE LISTING (84 sequences)

SEQ ID NO: 1 - protein, 252 aa, Influenza A virus
MSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSAGALASCMGLIYNRMGAVTTEVAFAVVCATCEQIADSQHRSHRQMVTTTNPLIRHENRMVLASTTAKAMEQMAGSSEQAAEAMEVASQARQMVQAMRAIGTPPSSSAGLKDDLLENLQAYQKRMGVQMQRFK

SEQ ID NO: 2 - protein, 9 aa, Artificial sequence
  The influenza virus derived peptide M1(58-66)
GILGFVFTL

SEQ ID NO: 3 - protein, 11 aa, Artificial sequence
  CDR1 amino acid of the influenza-M1 D12 light chain antibody
RASQSVSSNLA

SEQ ID NO: 4 - protein, 7 aa, Artificial sequence
  CDR2 amino acid of the influenza-M1 D12 light chain antibody
GASTRAT

SEQ ID NO: 5 - protein, 9 aa, Artificial sequence
  CDR3 amino acid of the influenza-M1 D12 light chain antibody
QQNYNWPLT

SEQ ID NO: 6 - protein, 5 aa, Artificial sequence
  CDR1 amino acid of the influenza-M1 D12 heavy chain antibody
SYAMH

SEQ ID NO: 7 - protein, 17 aa, Artificial sequence
  CDR2 amino acid of the influenza-M1 D12 heavy chain antibody
VISYDGSNKYYADSVKG

SEQ ID NO: 8 - protein, 16 aa, Artificial sequence
  CDR3 amino acid of the influenza-M1 D12 heavy chain antibody
DFWVSYYYDSSAPPAI

SEQ ID NO: 9 - DNA, 33 bp, Artificial sequence
  CDR1 nucleic acid of the influenza-M1 D12 light chain antibody
agggccagtcagagtgttagcagcaacttagcc

SEQ ID NO: 10 - DNA, 21 bp, Artificial sequence
  CDR2 nucleic acid of the influenza-M1 D12 light chain antibody
ggtgcatccaccagggccact

SEQ ID NO: 11 - DNA, 27 bp, Artificial sequence
  CDR3 nucleic acid of the influenza-M1 D12 light chain antibody
cagcagaattataactggcctctcact

SEQ ID NO: 12 - DNA, 15 bp, Artificial sequence
  CDR1 nucleic acid of the influenza-M1 D12 heavy chain antibody
agctatgctatgcac

SEQ ID NO: 13 - DNA, 51 bp, Artificial sequence
  CDR2 nucleic acid of the influenza-M1 D12 heavy chain antibody
gttatatcatatgatggaagcaataaatactacgcagactccgtgaagggc

SEQ ID NO: 14 - DNA, 48 bp, Artificial sequence
  CDR3 nucleic acid of the influenza-M1 D12 heavy chain antibody
gatttttgggtttcgtattactatgatagtagtgcccccccggctatc

SEQ ID NO: 15 - protein, 234 aa, Artificial sequence
  amino acid sequence of the influenza M1 D12 antibody light chain-CL
LETTLTQSPATLSVSPGERATLSCRASQSVSSNLAWYQQKPGQAPRLLIYGASTRATGIPARFSGSGSGTDFTLTISSLQSEDFAVYYCQQNYNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGECAPGSLHHILDAQKMVWNHR

SEQ ID NO: 16 - DNA, 702 bp, Artificial sequence
  nucleic acid sequence of the influenza M1 D12 antibody light chain-CL
cttgaaacgacactcacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagacttcactctcaccatcagcagcctgcagtctgaagactttgcagtttattactgtcagcagaattataactggcctctcactttcggcggcgggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgtgctcccgggtctctgcaccatatcctggacgcccagaagatggtgtggaatcaccgc

SEQ ID NO: 17 - protein, 125 aa, Artificial sequence
  amino acid sequence of the influenza M1 D12 antibody heavy chain
EVQLVQSGGGLVQPGGSLRLSCAASGFTFSSYAMHWVRQAPGKGLEWVAVISYDGSNKYYADSVKGRFTISRDNSKNTLYLQMNSLRAEDTAVYYCARDFWVSYYYDSSAPPAIWGQGTMVTVSS

SEQ ID NO: 18 - DNA, 375 bp, Artificial sequence
  nucleic acid sequence of the influenza M1 D12 antibody heavy chain
gaggtgcagctggtgcagtctgggggaggtttagttcagcctggggggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagagatttttgggtttcgtattactatgatagtagtgcccccccggctatctggggccaagggacaatggtcaccgtctcaagc

SEQ ID NO: 19 - protein, 97 aa, Influenza A virus
MSLLTEVETPIRNEWGCRCNGSSDPLAIAANIIGILHLILWILDRLFFKCIYRRFKYGLKGGPSTEGVPKSMREEYRKEQQSAVDADDGHFVSIELE

SEQ ID NO: 20 - protein, 716 aa, Influenza A virus
MEDFVRQCFNPMIVELAEKAMKEYGEDLKIETNKFAAICTHLEVCFMYSDFHFINEQGESIIVEPEDPNALLKHRFEIIEGRDRTMAWTVVNSICNTTGAEKPKFLPDLYDYKENRFIEIGVTRREVHIYYLEKANKIKSEKTHIHIFSFTGEEMATKADYTLDEESRARIKTRLFTIRQEMASRGLWDSFRQSERGEETIEERFEITGTMRRLADQSLPPNFSCLENFRAYVDGFEPNGYIEGKLSQMSKEVNARIEPFLRTTPRPIRLPDGPPCFQRSKFLLMDSLKLSIEDPSHEGEGIPLYDAIKCMRTFFGWKEPSVVKPHGKGINPNYLLSWKQVLAELQDIESEEKIPRTKNMKKTSQLKWALGENMAPEKVDFDDCKDISDLKQYDSDEPELRSLSSWIQNEFNKACELTDSIWIELDEIGEDVAPIEHIASMRRNYFTAEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLLRSAIGQVSRPMFLYVRTNGTSKIKMKWGMEMRRCLLQSLQQIESMIEAESSVKEKDMTKEFFENRSETWPIGESPKGVEEGSIGKVCRTLLAKSVFNSLYASPQLEGFSAESRKLLLIVQALRDNLEPGTFDLGGLYEAIEECLINDPWVLLNASWFNSFLTHALR

SEQ ID NO: 21 - protein, 248 aa, Influenza B virus
MSLFGDTIAYLLSLIEDGEGKAELAEKLHCWFGGKEFDLDSALEWIKNKRCLTDIQKALIGASICFLKPKDQERKRRFITEPLSGMGTTATKKKGLILAERKMRRCVSFHEAFEIAEGHESSALLYCLMVMYLNPENYSMQVKLGTLCALCEKQASHSHRAHSRAARSSVPGVRREMQMVSAMNTAKTMNGMGKGEDVQKLAEELQNNIGVLRSLGASQKNGEGIAKDVMEVLKQSSMGNSALVRKYL

SEQ ID NO: 22 - protein, 752 aa, Influenza B virus
MNINPYFLFIDVPIQAAISTTFPYTGVPPYSHGTGTGYTIDTVIRTHEYSNKGKQYISDVTGCVMVDPTNGPLPEDNEPSAYAQLDCVLEALDRMDEEHPGLFQAGSQNAMEALMVTTVDKLTQGRQTFDWTVCRNQPAATALNTTITSFRLNDLNGADKGGLVPFCQDIIDSLDKPEMIFFTVKNIKKKLPAKNRKGFLIKRIPMKVKDRITRVEYIKRALSLNTMTKDAERGKLKRRAIATAGIQIRGFVLVVENLAKNICENLEQSGLPVGGNEKKAKLSNAVAKMLSNCPPGGISMTVTGDNTKWNECLNPRIFLAMTERITRDSPIWFRDFCSIAPVLFSNKIARLGKGFMITSKTKRLKAQIPCPDLFNIPLERYNEETRAKLKKLKPFFNEEGTASLSPGMMMGMFNMLSTVLGVAALGIKNIGNKEYLWDGLQSSDDFALFVNAKDEETCMEGINDFYRTCKLLGINMSKKKSYCNETGMFEFTSMFYRDGFVSNFAMELPSFGVAGVNESADMAIGMTIIKNNMINNGMGPATAQTAIQLFIADYRYTYKCHRGDSKVEGKRMKIIKELWENTKGRDGLLVADGGPNLYNLRNLHIPEIILKYNIMDPEYKGRLLHPQNPFVGHLSIEGIKEADITPAHGPIKKMDYDAVSGTHSWRTKRNRSILNTDQRNMILEEQCYAKCCNLFEACFNSASYRKPVGQHSMLEAMAHRLRMDARLDYESGRMSKEDFEKAMAHLGEIGYM

SEQ ID NO: 23 - protein, 281 aa, Influenza B virus
MADNMTTTQIEVGPGATNATINFEAGILECYERFSWQRALDYPGQDRLHRLKRKLESRIKTHNKSEPENKRMSLEERKAIGVKMMKVLLFMDPSAGIEGFEPYCVKNPSTSKCPNYDWTDYPPTPGKYLDDIEEEPENVDHPIEVVLRDMNNKDARQKIKDEVNTQKEGKFRLTIKRDIRNVLSLRVLVNGTFLKHPNGDKSLSTLHRLNAYDQNGGLVAKLVATDDRTVEDEKDGHRILNSLFERFDEGHSKPIRAAETAVGVLSQFGQEHRLSPEEGDN

SEQ ID NO: 24 - protein, 759 aa, Influenza A virus
MERIKELRNLMSQSRTREILTKTTVDHMAIIKKYTSGRQEKNPALRMKWMMAMKYPITADKRITEMIPERNEQGQTLWSKMNDAGSDRVMVSPLAVTWWNRNGPMTNTVHYPKIYKTYFERVERLKHGTFGPVHFRNQVKIRRRVDINPGHADLSAKEAQDVIMEVVFPNEVGARILTSESQLTITKEKKEELQDCKISPLMVAYMLERELVRKTRFLPVAGGTSSVYIEVLHLTQGTCWEQMYTPGGEVKNDDVDQSLIIAARNIVRRAAVSADPLASLLEMCHSTQIGGIRMVDILKQNPTEEQAVGICKAAMGLRISSSFSFGGFTFKRTSGSSVKREEEVLTGNLQTLKIRVHEGYEEFTMVGRRATAILRKATRRLIQLIVSGRDEQSIAEAIIVAMVFSQEDCMIKAVRGDLNFVNRANQRLNPMHQLLRHFQKDAKVLFQNWGVEPIDNVMGMIGILPDMTPSIEMSMRGVRISKMGVDEYSSTERVVVSIDRFLRVRDQRGNVLLSPEEVSETQGTEKLTITYSSSMMWEINGPESVLVNTYQWIIRNWETVKIQWSQNPTMLYNKMEFEPFQSLVPKAIRGQYSGFVRTLFQQMRDVLGTFDTAQIIKLLPFAAAPPKQSRMQFSSFTVNVRGSGMRILVRGNSPVFNYNKATKRLTVLGKDAGTLTEDPDEGTAGVESAVLRGFLILGKEDRRYGPALSINELSNLAKGEKANVLIGQGDVVLVMKRKRDSSILTDSQTATKRIRMAIN

SEQ ID NO: 25 - protein, 109 aa, Influenza B virus
MLEPLQILSICSFILSALHFMAWTIGHLNQIKRGVNLKIQIRNPNKEAINREVSILRHNYQKEIQAKETMKKILSDNMEVLGDHIVVEGLSTDEIIKMGETVLEVEELQ

SEQ ID NO: 26 - protein, 122 aa, Influenza B virus
MADNMTTTQIEWRMKKMAIGSSTHSSSVLMKDIQSQFEQLKLRWESYPNLVKSTDYHQKRETIRLATEELYLLSKRIDDSILFHKTVIANSSIIADMIVSLSLLETLYEMKDVVEVYSRQCL

SEQ ID NO: 27 - protein, 100 aa, Influenza B virus
MNNATFNCTNINPITHIRGSIIITICVSLIVILIVFGCIAKIFINKNNCTNNVIRVHKRIKCPDCEPFCNKRDDISTPRAGVDIPSFILPGLNLSEGTPN

SEQ ID NO: 28 - protein, 498 aa, Influenza A virus
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEEIRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRDPGNAEFEDLTFLARSALILRGSVAHKSCLPACVYGPAVASGYDFEREGYSLVGIDPFRLLQNSQVYSLIRPNENPAHKSQLVWMACHSAAFEDLRVLSFIKGTKVVPRGKLSTRGVQIASNENMETMESSTLELRSRYWAIRTRSGGNTNQQRASAGQISIQPTFSVQRNLPFDRTTVMAAFTGNTEGRTSDMRTEIIRMMESARPEDVSFQGRGVFELSDEKAASPIVPSFDMSNEGSYFFGDNAEEYDN

SEQ ID NO: 29 - protein, 565 aa, Influenza C virus
MSDRRQNRKTPDEQRKANALIINENIEAYIAICKEVGLNGDEMLILENGIAIEKAIRICCDGKYQEKREKKAREAQRADSNFNADSIGIRLVKRAGSGTNITYHAVVELTSRSRIVQILKSHWGNELNRAKIAGKRLGFSALFASNLEAIIYQRGRNAARRNGSAELFTLTQGAGIETRYKWIMEKHIGIGVLIADAKGLINGKREGKRGVDANVKLRAGTTGSPLERAMQGIEKKAFPGPLRALARRVVKANYNDAREALNVIAEASLLLKPQITNKMTMPWCMWLAARLTLKDEFANFCAYAGRRAFEVFNIAMEKIGICSFQGTIMNDDEIESIEDKAQVLMMACFGLAYEDFSLVSAMVSHPLKLRNRMKIGNFRVGEKVSTVLSPLLRFTRWAEFAQRFALQANTSREGAQISNSAVFAVERKITTDVQRVEELLNKVQAHEDEPLQTLYKKVREQISIIGRNKSEIKEFLGSSMYDLNDQEKQNPINFRSGAHPFFFEFDPDYNPIRVKRPKKPIAKRNSNISRLEEEGMDENSEIGQAKKMKPLDQLTSTSSNIPGKN

SEQ ID NO: 30 - protein, 466 aa, Influenza B virus
MLPSTVQTLTLLLTSGGVLLSLYVSASLSYLLYSDVLLKFSSTKTTAPTMSLECTNASNAQTVNHSATKEMTFPPPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 31 - protein, 498 aa, Influenza A virus
MASQGTKRSYEQMETDGERQNATEIRASVGKMIDGIGRFYIQMCTELKLSDYEGRLIQNSLTIERMVLSAFDERRNKYLEEHPSAGKDPKKTGGPIYKRVDGKWMRELVLYDKEEIRRIWRQANNGDDATAGLTHMMIWHSNLNDTTYQRTRALVRTGMDPRMCSLMQGSTLPRRSGAAGAAVKGVGTMVMELIRMIKRGINDRNFWRGENGRKTRSAYERMCNILKGKFQTAAQRAMMDQVRESRNPGNAEIEDLIFLARSALILRGSVAHKSCLPACVYGPAIASGYNFEKEGYSLVGIDPFKLLQNSQVYSLIRPNENPAHKSQLVWMACNSAAFEDLRVLSFIRGTKVSPRGKLSTRGVQIASNENMDTMESSTLELRSRYWAIRTRSGGNTNQQRASAGQISVQPAFSVQRNLPFDKPTIMAAFTGNTEGRTSDMRAEIIRMMEGAKPEEMSFQGRGVFELSDEKATNPIVPSFDMSNEGSYFFGDNAEEYDN

SEQ ID NO: 32 - protein, 584 aa, Influenza B virus
MKAIIVLLMVVTSNADRICTGITSSNSPHVVKTATQGEVNVTGVIPLTTTPTKSHFANLKGTQTRGKLCPNCFNCTDLDVALGRPKCMGNTPSAKVSILHEVKPATSGCFPIMHDRTKIRQLPNLLRGYENIRLSTSNVINTETAPGGPYKVGTSGSCPNVANGNGFFNTMAWVIPKDNNKTAINPVTVEVPYICSEGEDQITVWGFHSDDKTQMERLYGDSNPQKFTSSANGVTTHYVSQIGGFPNQTEDEGLKQSGRIVVDYMVQKPGKTGTIVYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNYLSELEVKNLQRLSGAMNELHDEILELDEKVDDLRADTISSQIELAVLLSNEGIINSEDEHLLALERKLKKMLGPSAVEIGNGCFETKHKCNQTCLDRIAAGTFNAGDFSLPTFDSLNITAASLNDDGLDNHTILLYYSTAASSLAVTLMIAIFIVYMVSRDNVSCSICL

SEQ ID NO: 33 - protein, 560 aa, Influenza B virus
MSNMDIDSINTGTIDKTPEELTPGTSGATRPIIKPATLAPPSNKRTRNPSPERTTTSSETDIGRKIQKKQTPTEIKKSVYKMVVKLGEFYNQMMVKAGLNDDMERNLIQNAQAVERILLAATDDKKTEYQKKRNARDVKEGKEEIDHNKTGGTFYKMVRDDKTIYFSPIKITFLKEEVKTMYKTTMGSDGFSGLNHIMIGHSQMNDVCFQRSKGLKRVGLDPSLISTFAGSTLPRRSGTTGVAIKGGGTLVDEAIRFIGRAMADRGLLRDIKAKTAYEKILLNLKNKCSAPQQKALVDQVIGSRNPGIADIEDLTLLARSMVVVRPSVASKVVLPISIYAKIPQLGFNTEEYSMVGYEAMALYNMATPVSILRMGDDAKDKSQLFFMSCFGAAYEDLRVLSALTGTEFKPRSALKCKGFHVPAKEQVEGMGAALMSIKLQFWAPMTRSGGNEVSGEGGSGQISCSPVFAVERPIALSKQAVRRMLSMNVEGRDADVKGNLLKMMNDSMAKKTSGNAFIGKKMFQISDKNKVNPIEIPIKQTIPNFFFGRDTAEDYDDLDY

SEQ ID NO: 34 - protein, 498 aa, Influenza A virus
MASQGTKRSYEQMETGGERQNATEIRASVGRMVGGIGRFYVQMCTELKLSDQEGRLIQNSITIERMVLSAFDERRNRYLEEHPSAGKDPKKTGGPIYRRRDGKWVRELILYDKEEIRRIWRQANNGEDATAGLTHMMIWHSNLNDATYQRTRALVRTGMDPRMCSLMQGSTLPRRSGAAGAAIKGVGTMVMELIRMIKRGINDRNFWRGDNGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVIPRGQLSTRGVQIASNENVEAMDSSTLELRSRYWAIRTRSGGNTNQQRASAGQISVQPTFSVQRNLPFERPTIMAAFKGNTEGRTSDMRTEIIRMMESARPEDVSFQGRGVFELSDEKATNPIVPSFDMSNEGSYFFGDNAEEYDN

SEQ ID NO: 35 - protein, 566 aa, Influenza A virus
MKTIIALSYILCLVFAQKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSACKRRSNNSFFSRLNWLTHLKFKYPALNVTMPNNEKFDKLYIWGVHHPGTDNDQISLYAQASGRITVSTKRSQQTVIPSIGSRPRIRDVPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 36 - protein, 9 aa, Artificial sequence
  HLA-A2-restricted hTERT (540) peptide
ILAKFLHWL

SEQ ID NO: 37 - protein, 9 aa, Artificial sequence
  HLA-A2-restricted hTERT (865) peptide
RLVDDFLLV

SEQ ID NO: 38 - protein, 10 aa, Artificial sequence
  HLA-A2-restricted MART-1 peptide
ELAGIGILTV

SEQ ID NO: 39 - protein, 9 aa, Artificial sequence
  HLA-A2-restricted EBV peptide
GLCTLVAML

SEQ ID NO: 40 - protein, 9 aa, Artificial sequence
  HLA-A2-restricted CMV peptide
NLVPMVATV

SEQ ID NO: 41 - protein, 9 aa, Artificial sequence
  HLA-A2-restricted gp100 (G9 209-2M)  peptide
IMDQVPFSV

SEQ ID NO: 42 - protein, 9 aa, Artificial sequence
  HLA-A2-restricted gp100 (280) peptide
YLEPGPVTA

SEQ ID NO: 43 - protein, 9 aa, Artificial sequence
  HLA-A2-restricted gp100 (154) peptide
KTWGQYWQV

SEQ ID NO: 44 - protein, 9 aa, Artificial sequence
  HLA-A2-restricted HTLV (TAX)  peptide
LLFGYPVYV

SEQ ID NO: 45 - protein, 9 aa, Artificial sequence
  HLA-A2-restricted MUC1 13-21 peptide
LLLTVLTVL

SEQ ID NO: 46 - protein, 15 aa, Artificial sequence
  a peptide for site specific biotinylation
LHHILDAQKMVWNHR

SEQ ID NO: 47 - DNA, 23 bp, Artificial sequence
  Single strand DNA oligonucleotide
agcggataacaatttcacacagg

SEQ ID NO: 48 - DNA, 24 bp, Artificial sequence
  Single strand DNA oligonucleotide
tttgtcgtctttccagacgttagt

SEQ ID NO: 49 - protein, 238 aa, Aequorea macrodactyla
MSKGEELFTGIVPVLIELDGDVHGHKFSVRGEGEGDADYGKLEIKFICTTGKLPVPWPTLVTTLGYGIQCFARYPEHMKMNDFFKSAMPEGYIQERTIFFQDDGKYKTRGEVKFEGDTLVNRIELKGMDFKEDGNILGHKLEYNFNSHNVYIMPDKANNGLKVNFKIRHNIEGGGVQLADHYQTNVPLGDGPVLIPINHYLSLQTAISKDRNETRDHMVFLEFFSACGHTHGMDELYK

SEQ ID NO: 50 - DNA, 717 bp, Aequorea macrodactyla
atgagtaaaggagaagaacttttcactgggattgtcccagttctcattgagttagacggtgatgtccatggacataaattctctgtcagaggagaaggggaaggcgatgcagattatggaaaacttgaaatcaaattcatttgcactactggaaagctaccagttccatggccaacacttgttactacactgggctacggcatccaatgtttcgcaagatacccagaacacatgaaaatgaatgacttcttcaagagtgccatgcctgagggttacattcaagaaagaaccatctttttccaagatgatggaaaatacaagacacgtggtgaagtcaagtttgaaggtgatactcttgttaacagaattgagctcaaaggtatggactttaaagaagatggcaatatccttggacacaagttggagtacaattttaattcacataatgtatacattatgccggacaaagccaataatggactcaaagtcaatttcaaaattagacacaatatcgaaggtggtggtgtccaacttgctgatcattaccaaacaaatgttccccttggagacggtcctgtccttataccaatcaatcactacctatccttgcaaacagccatttcaaaagatcgaaatgagacgagagatcatatggtgtttctggaatttttctcagcttgtggacatacacatggcatggatgaactatacaaataa

SEQ ID NO: 51 - protein, 489 aa, Artificial sequence
  alkaline phosphatase
MKQSTIALALLPLLFTPVTKARTPEMPLQGTAVDGGGGSMHASLEVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 52 - DNA, 1470 bp, Artificial sequence
  alkaline phosphatase
ttatttcagccccagagcggctttcatggtgtagaagagatcggtctggtcggtcagtccaacaacattggcggcatgcgggccatacgccgcaatacgcaactgactgccggtatgttcttgtgaatcctcttcggagttcccgtaactcatcaccatcactgcgccatctttggtatttagcgcctgggtgaggcccggagctttggtatccggcgcaacaatctggctggcgtgggcgtgatcagcggtgactatgaccagcgtgttaccctcctttttagcgaattccagcgcccgttgtacggcttcatcgagatcgaccgtctcgccaatttgcccacaaggattcgcagcatgatcctgtttatcgattgacgcaccttcaacttgcaggaaaaagcctttctcatttttactcaacaattcaatggctttgtcggtcatctgcgccagggttggtacactgtcattacgttgcggatttggcgtacaggtgactgcgggcttatcgatattgccatggtacgttgctttcggtcctagccagcgcactggcatattgccgtcagcaaacaggccaagcaggggtttttgctgattcgcttccgtcaccgaattcagtgaggcagcatcgctcaccaactgataaccacgcgcctgtgcctgttcacgcagcgtttttccctgccattcaccagcggttgccgtttcagcaaaggtttttgcgccgccgccaagcgtaacgtcggcacgagcgttaagcagctgttcggtaatcgatccttttccgcctttttccagagcgttacccggacatttttcactggtcgcgctcggaccgtagcatttgcgcgaggtcacatgtgccaccagcgcagcgggcgtggcatcctgcaactctgcggtagaaacgttaccggtcgccagacctgcggcttttgccatttccagaatcgttgggtgatctttttcgtgaatatcgacgcccagcgcgccgttataggttttgacaccggttgaccaggcggttgctgatgcagccgagtcggtgacgtagtccggtttgccggtttttttattcagcgcatagtgagtgtattgcccggtaagcggtaaggcatctatacctttaaaaaagccgcccgcaccttcggcataattacgtgcggcagtaatttccgagtcccccatcccatcgccaatcagcaaaataatattttttgcaggtttatcgctaagagaatcacgcagagcggcagtctgatcacccgttaaacggcgagcaccgccgggtgcagtaatatcgccctgagcagcccggttttccagaacctcgaggctagcatgcatagaaccgccaccaccgtcgacagcggtaccctgcagaggcatttctggtgtccgggcttttgtcacaggggtaaacagtaacggtaagagtgccagtgcaatagtgctttgtttcac

SEQ ID NO: 53 - protein, 309 aa, Artificial sequence
  Horseradish peroxidase
MQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNS

SEQ ID NO: 54 - DNA, 955 bp, Artificial sequence
  Horseradish peroxidase
aagcttaaccatgcagttaacccctacattctacgacaatagctgtcccaacgtgtccaacatcgttcgcgacacaatcgtcaacgagctcagatccgatcccaggatcgctgcttcaatattacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctgactatagctgcgcaacagagcgtgactcttgcaggcggaccgtcctggagagtgccgctcggtcgacgtgactccctacaggcattcctagatctggccaacgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatctgcggaccccaaccatcttcgataacaagtactatgtgaatctagaggagcagaaaggcctgatacagagtgatcaagaactgtttagcagtccaaacgccactgacaccatcccactggtgagaagttttgctaactctactcaaaccttctttaacgccttcgtggaagccatggaccgtatgggtaacattacccctctgacgggtacccaaggccagattcgtctgaactgcagagtggtcaacagcaactcttaataaggatccgaattc

SEQ ID NO: 55 - protein, 6 aa, Artificial sequence
  poly-histidine tag
HHHHHH

SEQ ID NO: 56 - DNA, 15 bp, Artificial sequence
  poly-histidine tag
catcatcatcaccat

SEQ ID NO: 57 - protein, 11 aa, Artificial sequence
  c-myc tag
EQKLISEEDLN

SEQ ID NO: 58 - DNA, 33 bp, Artificial sequence
  c-myc tag
gaacaaaaactcatctcagaagaggatctgaat

SEQ ID NO: 59 - protein, 238 aa, Artificial sequence
  orange fluorescent protein
MSKGEELFTGVVPILVELDGDVHGHKFSVRGEGEGDADYGKLEIKFICTTGKLPVPWPTLVTTLGYGILCFARYPEHMKMNDFFKSAMPEGYIQERTIFFQDDGKYKTRGEVKFEGDTLVNRIELKGMDFKEDGNILGHKLEYNFNSHNVYIMPDKANNGLKVNFKIRHNIEGGGVQLADHYQTNVPLGDGPVLIPINHYLSYQTAISKDRNETRDHMVFLEFFSACGHTHGMDELYK

SEQ ID NO: 60 - DNA, 717 bp, Artificial sequence
  orange fluorescent protein
atgagtaaaggagaagaacttttcactggagttgtcccaattcttgttgaattagatggtgatgtccatggacataaattctctgtcagaggagaaggggaaggcgatgcagattatggaaaacttgaaatcaaattcatttgcactactggaaagctaccagttccatggccaacacttgttactacactgggctatggcatcctatgtttcgcaagatacccagaacacatgaaaatgaatgacttcttcaagagtgccatgcctgagggttacattcaagaaagaaccatctttttccaagatgatggaaaatacaagacacgtggtgaagtcaagtttgaaggtgatactcttgttaacagaattgagctcaaaggtatggactttaaagaagatggcaatatccttggacacaagttggagtacaattttaactcacataatgtatacattatgccggacaaagccaataatggactcaaagtcaatttcaaaattagacacaatatcgaaggtggtggtgtccaactcgctgatcattaccaaacaaatgttccccttggagacggtcctgtccttataccaatcaatcactacctatcctatcaaacagccatttcaaaagatcgaaatgagacgagagatcatatggtgtttctggaatttttctcagcttgtggacatacacatggcatggatgaactatacaaataa

SEQ ID NO: 61 - protein, 1019 aa, Artificial sequence
  Beta galactosidase
MADPVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSADFQLSAGRYHYQLVWCQK

SEQ ID NO: 62 - DNA, 3060 bp, Artificial sequence
  Beta galactosidase
ttatttttgacaccagaccaactggtaatggtagcgaccggcgctcagctggaaatccgccgatactgacgggctccaggagtcgtcgccaccaatccccatatggaaaccgtcgatattcagccatgtgccttcttccgcgtgcagcagatggcgatggctggtttccatcagttgctgttgactgtagcggctgatgttgaactggaagtcgccgcgccactggtgtgggccataattcaattcgcgcgtcccgcagcgcagaccgttttcgctcgggaagacgtacggggtatacatgtctgacaatggcagatcccagcggtcaaaacaggcggcagtaaggcggtcgggatagttttcttgcggccctaatccgagccagtttacccgctctgctacctgcgccagctggcagttcaggccaatccgcgccggatgcggtgtatcgctcgccacttcaacatcaacggtaatcgccatttgaccactaccatcaatccggtaggttttccggctgataaataaggttttcccctgatgctgccacgcgtgagcggtcgtaatcagcaccgcatcagcaagtgtatctgccgtgcactgcaacaacgctgcttcggcctggtaatggcccgccgccttccagcgttcgacccaggcgttagggtcaatgcgggtcgcttcacttacgccaatgtcgttatccagcggtgcacgggtgaactgatcgcgcagcggcgtcagcagttgttttttatcgccaatccacatctgtgaaagaaagcctgactggcggttaaattgccaacgcttattacccagctcgatgcaaaaatccatttcgctggtggtcagatgcgggatggcgtgggacgcggcggggagcgtcacactgaggttttccgccagacgccactgctgccaggcgctgatgtgcccggcttctgaccatgcggtcgcgttcggttgcactacgcgtactgtgagccagagttgcccggcgctctccggctgcggtagttcaggcagttcaatcaactgtttaccttgtggagcgacatccagaggcacttcaccgcttgccagcggcttaccatccagcgccaccatccagtgcaggagctcgttatcgctatgacggaacaggtattcgctggtcacttcgatggtttgcccggataaacggaactggaaaaactgctgctggtgttttgcttccgtcagcgctggatgcggcgtgcggtcggcaaagaccagaccgttcatacagaactggcgatcgttcggcgtatcgccaaaatcaccgccgtaagccgaccacgggttgccgttttcatcatatttaatcagcgactgatccacccagtcccagacgaagccgccctgtaaacggggatactgacgaaacgcctgccagtatttagcgaaaccgccaagactgttacccatcgcgtgggcgtattcgcaaaggatcagcgggcgcgtctctccaggtagcgaaagccattttttgatggaccatttcggcacagccgggaagggctggtcttcatccacgcgcgcgtacatcgggcaaataatatcggtggccgtggtgtcggctccgccgccttcatactgcaccgggcgggaaggatcgacagatttgatccagcgatacagcgcgtcgtgattagcgccgtggcctgattcattccccagcgaccagatgatcacactcgggtgattacgatcgcgctgcaccattcgcgttacgcgttcgctcatcgccggtagccagcgcggatcatcggtcagacgattcattggcaccatgccgtgggtttcaatattggcttcatccaccacatacaggccgtagcggtcgcacagcgtgtaccacagcggatggttcggataatgcgaacagcgcacggcgttaaagttgttctgcttcatcagcaggatatcctgcaccatcgtctgctcatccatgacctgaccatgcagaggatgatgctcgtgacggttaacgcctcgaatcagcaacggcttgccgttcagcagcagcagaccattttcaatccgcacctcgcggaaaccgacatcgcaggcttctgcttcaatcagcgtgccgtcggcggtgtgcagttcaaccaccgcacgatagagattcgggatttcggcgctccacagtttcgggttttcgacgttcagacgtagtgtgacgcgatcggcataaccaccacgctcatcgataatttcaccgccgaaaggcgcggtgccgctggcgacctgcgtttcaccctgccataaagaaactgttacccgtaggtagtcacgcaactcgccgcacatctgaacttcagcctccagtacagcgcggctgaaatcatcattaaagcgagtggcaacatggaaatcgctgatttgtgtagtcggtttatgcagcaacgagacgtcacggaaaatgccgctcatccgccacatatcctgatcttccagataactgccgtcactccaacgcagcaccatcaccgcgaggcggttttctccggcgcgtaaaaatgcgctcaggtcaaattcagacggcaaacgactgtcctggccgtaaccgacccagcgcccgttgcaccacagatgaaacgccgagttaacgccatcaaaaataattcgcgtctggccttcctgtagccagctttcatcaacattaaatgtgagcgagtaacaacccgtcggattctccgtgggaacaaacggcggattgaccgtaatgggataggttacgttggtgtagatgggcgcatcgtaaccgtgcatctgccagtttgaggggacgacgacagtatcggcctcaggaagatcgcactccagccagctttccggcaccgcttctggtgccggaaaccaggcaaagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacgggatcagccat

SEQ ID NO: 63 - protein, 159 aa, Artificial sequence
  streptavidin
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 64 - DNA, 483 bp, Artificial sequence
  streptavidin
gacccgagcaaagattctaaagcacaagtatctgctgcagaagcaggaattacaggcacatggtataatcagctgggatctacatttattgttacagccggcgcagatggagctcttacaggaacatatgaatctgctgttggaaatgcagaatctagatacgtgcttacaggaagatatgattctgcacctgcaacagatggatccggaacagcacttggatggacagttgcatggaaaaacaattatagaaacgcacatagcgctacaacatggtctggccaatatgtgggaggtgcagaagcaagaattaacacacaatggcttttaacatctggaacaacagaagcaaatgcatggaaaagtactcttgttggacatgatacatttacaaaagttaaacctagcgcagcatctatcgatgcagcgaaaaaagcaggagttaacaatggcaatcctttagatgcagttcaacaataatga

SEQ ID NO: 65 - protein, 216 aa, Artificial sequence
  Pseudomonas exotoxin A
AEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLAPSSIPDQEQAISALPDYASQPGKPSREDLK

SEQ ID NO: 66 - DNA, 651 bp, Artificial sequence
  Pseudomonas exotoxin A
gcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggaccttgacgcgatctggcgcggtttctatatcgccggcgatccggcgctggcctacggctacgcccaggaccaggaacccgacgcgcgcggccggatccgcaacggtgccctgctgcgggtctatgtgccgcgctcgagtctgccgggcttctaccgcaccggcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccacgcaacgtcggcggcgacctcgccccgtccagcatccccgaccaggaacaggcgatcagcgccctgccggactacgccagccagcccggcaaaccgtcgcgcgaggacctgaagtaa

SEQ ID NO: 67 - protein, 536 aa, Artificial sequence
  diphtheria toxin
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 68 - DNA, 1611 bp, Artificial sequence
  diphtheria toxin
atgggcgctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatcagtaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcatctttaatggttgctcaagctattccattggtaggagagctagttgatattggtttcgctgcatataattttgtagagagtattatcaatttatttcaagtagttcataattcgtataatcgtcccgcgtattctccggggcataaaacgcaaccatttcttcatgacgggtatgctgtcagttggaacactgttgaagattcgataatccgaactggttttcaaggggagagtgggcacgacataaaaattactgctgaaaataccccgcttccaatcgcgggtgtcctactaccgactattcctggaaagctggacgttaataagtccaagactcatatttccgtaaatggtcggaaaataaggatgcgttgcagagctatagacggtgatgtaactttttgtcgccctaaatctcctgtttatgttggtaatggtgtgcatgcgaatcttcacgtggcatttcacagaagcagctcggagaaaattcattctaatgaaatttcatcggattccataggcgttcttgggtaccagaaaacagtagatcacaccaaggttaattctaagctatcgctattttttgaaatcaaaagctga

SEQ ID NO: 69 - protein, 127 aa, Artificial sequence
  interleukin 2 (IL-2)
TKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 70 - DNA, 698 bp, Artificial sequence
  interleukin 2 (IL-2)
gggngggggacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaacaaaaaaaaaaaaaccccccccc

SEQ ID NO: 71 - protein, 207 aa, Artificial sequence
  CD3
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 72 - DNA, 1311 bp, Artificial sequence
  CD3
gtaagtctgctggcctccgccatcttagtaaagtaacagtcccatgaaacaaagatgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgaccctctggagaacactgcctcccgctggcccaggtctcctctccagtccccctgcgactccctgtttcctgggctagtcttggaccccacgagagagaatcgttcctcagcctcatggtgaactcgcgccctccagcctgatcccccgctccctcctccctgccttctctgctggtacccagtcctaaaatattgctgcttcctcttcctttgaagcatcatcagtagtcacaccctcacagctggcctgccctcttgccaggatatttatttgtgctattcactcccttccctttggatgtaacttctccgttcagttccctccttttcttgcatgtaagttgtcccccatcccaaagtattccatctacttttctatcgccgtccccttttgcagccctctctggggatggactgggtaaatgttgacagaggccctgccccgttcacagatcctggccctgagccagccctgtgctcctccctcccccaacactccctaccaaccccctaatcccctactccctccaaccccccctcccactgtaggccactggatggtcatttggcatctccgtatatgtgctctggctcctcagctgagagagaaaaaaataaactgtatttggctgc

SEQ ID NO: 73 - protein, 290 aa, Artificial sequence
  CD16
MGGGAGERLFTSSCLVGLVPLGLRISLVTCPLQCGIMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 74 - DNA, 2406 bp, Artificial sequence
  CD16
gattctgtgtgtgtcctcagatgctcagccacagacctttgagggagtaaagggggcagacccacccaccttgcctccaggctctttccttcctggtcctgttctatggtggggctcccttgccagacttcagactgagaagtcagatgaagtttcaagaaaaggaaattggtgggtgacagagatgggtggaggggctggggaaaggctgtttacttcctcctgtctagtcggtttggtccctttagggctccggatatctttggtgacttgtccactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccccatcatcctcaggcctctctacaagcagcaggaaacatagaactcagagccagatcccttatccaactctcgacttttccttggtctccagtggaagggaaaagcccatgatcttcaagcagggaagccccagtgagtagctgcattcctagaaattgaagtttcagagctacacaaacactttttctgtcccaaccgttccctcacagcaaagcaacaatacaggctagggatggtaatcctttaaacatacaaaaattgctcgtgttataaattacccagtttagaggggaaaaaaaaacaattattcctaaataaatggataagtagaattaatggttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatatctaattcctgtgtagctttgttcattgcatttattaaacaaatgttgtataaccaatactaaatgtactactgagcttcgctgagttaagttatgaaactttcaaatccttcatcatgtcagttccaatgaggtggggatggagaagacaattgttgcttatgaaagaaagctttagctgtctctgttttgtaagctttaagcgcaacatttcttggttccaataaagcattttacaagatcttgcatgctactcttagatagaagatgggaaaaccatggtaataaaatatgaatgataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 75 - protein, 153 aa, Artificial sequence [sequence omitted]

SEQ ID NO: 76 - DNA, 921 bp, Artificial sequence
  IL-4
ttctatgcaaagcaaaaagccagcagcagccccaagctgataagattaatctaaagagcaaattatggtgtaatttcctatgctgaaactttgtagttaattttttaaaaaggtttcattttcctattggtctgatttcacaggaacattttacctgtttgtgaggcattttttctcctggaagagaggtgctgattggccccaagtgactgacaatctggtgtaacgaaaatttccaatgtaaactcattttccctcggtttcagcaattttaaatctatatatagagatatctttgtcagcattgcatcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatct

SEQ ID NO: 77 - protein, 365 aa, Artificial sequence [sequence omitted]

SEQ ID NO: 78 - DNA, 4000 bp, Artificial sequence
  HLA class I histocompatibility antigen, A-2 alpha chain
aagcttactctctggcaccaaactccatgggatgatttttccttcctagaagagtccaggtggacaggtaaggagtgggagtcagggagtccagttccagggacagagattacgggataaaaagtgaaaggagagggacggggcccatgccgagggtttctcccttgtttctcagacagctcttgggccaagactcagggagacattgagacagagcgcttggcacagaagcagaggggtcagggcgaagtccagggccccaggcgttggctctcagggtctcaggccccgaaggcggtgtatggattggggagtcccagccttggggattccccaactccgcagtttcttttctccctctcccaacctatgtagggtccttcttcctggatactcacgacgcggacccagttctcactcccattgggtgtcgggtttccagagaagccaatcagtgtcgtcgcggtcgcggttctaaagtccgcacgcacccaccgggactcagattctccccagacgccgaggatggccgtcatggcgccccgaaccctcgtcctgctactctcgggggctctggccctgacccagacctgggcgggtgagtgcggggtcgggagggaaacggcctctgtggggagaagcaacgggccgcctggcgggggcgcaggacccgggaagccgcgccgggaggagggtcgggcgggtctcagccactcctcgtccccaggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggtgagtgaccccggcccggggcgcaggtcacgacctctcatcccccacggacgggccaggtcgcccacagtctccgggtccgagatccgccccgaagccgcgggaccccgagacccttgccccgggagaggcccaggcgcctttacccggtttcattttcagtttaggccaaaaatccccccaggttggtcggggcggggcggggctcgggggaccgggctgaccgcggggtccgggccaggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacgggtaccaggggccacggggcgcctccctgatcgcctgtagatctcccgggctggcctcccacaaggaggggagacaattgggaccaacactagaatatcgccctccctctggtcctgagggagaggaatcctcctgggtttccagatcctgtaccagagagtgactctgaggttccgccctgctctctgacacaattaagggataaaatctctgaaggaatgacgggaagacgatccctcgaatactgatgagtggttccctttgacacacacaggcagcagccttgggcccgtgacttttcctctcaggccttgttctctgcttcacactcaatgtgtgtgggggtctgagtccagcacttctgagtccttcagcctccactcaggtcaggaccagaagtcgctgttccctcttcagggactagaatttccacggaataggagattatcccaggtgcctgtgtccaggctggtgtctgggttctgtgctcccttccccatcccaggtgtcctgtccattctcaagatagccacatgtgtgctggaggagtgtcccatgacagatcgaaaatgcctgaatgatctgactcttcctgacagacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatggggtaaggagggagacgggggtgtcatgtcttttagggaaagcaggagcctctctgacctttagcagggtcagggcccctcaccttcccctcttttcccagagccgtcttcccagcccaccatccccatcgtgggcatcattgctggcctggttctctttggagctgtgatcactggagctgtggtcgctgctgtgatgtggaggaggaagagctcaggtggggaaggggtgaagggtgggtctgagatttcttgtctcactgagggttccaagacccaggtagaagtgtgccctgcctcgttactgggaagcaccacccacaattatgggcctacccagcctgggccctgtgtgccagcacttactcttttgtaaagcacctgttaaaatgaaggacagatttatcaccttgattacagcggtgatgggacctgatcccagcagtcacaagtcacaggggaaggtccctgaggaccttcaggagggcggttggtccaggacccacacctgctttcttcatgtttcctgatcccgccctgggtctgcagtcacacatttctggaaacttctctgaggtccaagacttggaggttcctctaggaccttaaggccctgactcttttctggtatctcacaggacattttcttcccacagatagaaaaggagggagctactctcaggctgcaagtaagtatgaaggaggctgatgcctgaggtccttgggatattgtgtttgggagcccatgggggagctcacccaccccacaattcctcctctagccacatcttctgtgggatctgaccaggttctgtttttgttctaccccaggcagtgacagtgcccagggctctgatgtgtctctcacagcttgtaaaggtgagagcctggagggcctgatgtgtgttgggtgttgggcggaacagtggacacagctgtgctatggggtttctttccattggatgtattgagcatgcgatgggctgtttaaagtgtgacccctcactgtgacagatacgaatttgttcatgaatatttttttctatagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagaccaccccacccccatgtccaccatgaccctcttcccacgctgacctgtgctccctccccaatcatctttcctgttccagagaggtggggctgaggtgtctccatctctgtctcaacttcatggtgcactgagctgtaacttcttccttccctattaaaattagaacctgagtataaatttactttctcaaattcttgccatgagaggttgatgagttaattaaaggagaagattcctaaaatttgagagacaaaataaatggaacacatgagaaccttccagagtccacgtgttgcttatgctgatttgttgcaggggaggagagtagatggggctgtgcccagtttctgttccggccactatgggctttatgtggtcactgcttggctgggtcatctttgctgctccattgtccttgg

SEQ ID NO: 79 - protein, 178 aa, Artificial sequence
  Interleukin-10
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 80 - DNA, 1601 bp, Artificial sequence
  Interleukin-10
aaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 81 - protein, 576 aa, Artificial sequence
  Ricin
MKPGGNTIVIWMYAVATWLCFGSTSGWSFTLEDNNIFPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQFSLLIRPVVPNFNADVCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRASDPSLKQIILYPLHGDPNQIWLPLF

SEQ ID NO: 82 - DNA, 1887 bp, Artificial sequence
  Ricin
atgaaaccgggaggaaatactattgtaatatggatgtatgcagtggcaacatggctttgttttggatccacctcagggtggtctttcacattagaggataacaacatattccccaaacaatacccaattataaactttaccacagcgggtgccactgtgcaaagctacacaaactttatcagagctgttcgcggtcgtttaacaactggagctgatgtgagacatgaaataccagtgttgccaaacagagttggtttgcctataaaccaacggtttattttagttgaactctcaaatcatgcagagctttctgttacattagcgctggatgtcaccaatgcatatgtggtcggctaccgtgctggaaatagcgcatatttctttcatcctgacaatcaggaagatgcagaagcaatcactcatcttttcactgatgttcaaaatcgatatacattcgcctttggtggtaattatgatagacttgaacaacttgctggtaatctgagagaaaatatcgagttgggaaatggtccactagaggaggctatctcagcgctttattattacagtactggtggcactcagcttccaactctggctcgttcctttataatttgcatccaaatgatttcagaagcagcaagattccaatatattgagggagaaatgcgcacgagaattaggtacaaccggagatctgcaccagatcctagcgtaattacacttgagaatagttgggggagactttccactgcaattcaagagtctaaccaaggagcctttgctagtccaattcaactgcaaagacgtaatggttccaaattcagtgtgtacgatgtgagtatattaatccctatcatagctctcatggtgtatagatgcgcacctccaccatcgtcacagttttctttgcttataaggccagtggtaccaaattttaatgctgatgtttgtatggatcctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtaccacacttacagtgcaaaccaacatttatgccgttagtcaaggttggcttcctactaataatacacaaccttttgtgacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgtcaagatcctctcttgtggccctgcatcctctggccaacgatggatgttcaagaatgatggaaccattttaaatttgtatagtgggttggtgttagatgtgagggcatcggatccgagccttaaacaaatcattctttaccctctccatggtgacccaaaccaaatatggttaccattattttgatagacagattactctcttgcagtgtgtatgtcctgccatgaaaatagatggcttaaataaaaaggacattgtaaattttgtaactgaaaggacagcaagttattgcagtccagtatctaataagagcacaactattgtcttgtgcattctaaattt

SEQ ID NO: 83 - protein, 353 aa, Artificial sequence
  PE38KDEL polypeptide
AAASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 84 - DNA, 1059 bp, Artificial sequence
  PE38KDEL nucleotide sequence
gcggccgcttccggaggtcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaag

(19) United States
(12) Patent Application Publication
(10) Pub. No.: US 2011/0033473 A1
(43) Pub. Date: Feb. 10, 2011

(54) ANTI INFLUENZA ANTIBODIES AND USES THEREOF

(76) Inventors: Yoram Reiter, Haifa (IL); Avital Lev, Dresher (IL)

Correspondence Address: MARTIN D. MOYNIHAN d/b/a PRTSI, INC., P.O. BOX 16446, ARLINGTON, VA, 22215, US

(21) Appl. No.: 12/936,518

(22) PCT Filed: Apr. 5, 2009
(86) PCT No.: PCT/IL09/00380

§ 371 Date: Oct. 6, 2010

Related U.S. Application Data
(60) Provisional application No. 61/071,044, filed on Apr. 9, 2008.

(51) Int. Cl. A61K 39/42; C07K 16/10; C07H 21/00; C12N 15/63; C12Q 1/70; A61K 31/7088; A61P 31/16

(52) U.S. Cl. 424/139.1; 530/387.9; 530/387.3; 536/23.53; 435/320.1; 435/5; 514/44 R

(57) ABSTRACT

Provided are antibodies comprising an antigen recognition domain capable of binding an MHC molecule being complexed with an influenza peptide wherein the antibody does not bind the MHC molecule in an absence of the complexed peptide and wherein the antibody does not bind the peptide in an absence of the MHC molecule. Also provided are methods of using same for diagnosing and treating influenza.

### FIELD AND BACKGROUND OF THE INVENTION

The present invention, in some embodiments thereof, relates to antibodies which can specifically bind complexes of MHC and an influenza antigenic peptide and uses thereof in the diagnosis and treatment of influenza infection in a subject.

Influenza is an acute febrile illness caused by infection of the respiratory tract. The disease can cause significant systemic symptoms, severe illness requiring hospitalization (such as viral pneumonia), and complications such as secondary bacterial pneumonia. Influenza viruses cause epidemics of disease almost every winter in all countries and are a leading cause of death in the developed world. In the United States, the winter influenza epidemics can cause illness in 10-20% of the population and are associated with an average of 20,000 deaths and 114,000 hospitalizations per year.

There are three types of influenza viruses: A, B, and C. Type A, which includes several subtypes, causes widespread epidemics and global pandemics such as those that occurred in 1918, 1957 and 1968; type B causes regional epidemics; and type C causes sporadic cases and minor, local outbreaks. The virus types are distinguished, in part, on the basis of differences in two structural proteins, the nucleoprotein, found in the center of the virus, and the matrix protein, which forms the viral shell.

The control of influenza involves yearly vaccination, especially of people in high-risk groups, such as residents of nursing or residential homes, and those having diabetes, chronic renal failure, or chronic respiratory conditions.

The currently available influenza vaccines include whole virus or subvirion vaccines. The whole virus vaccine contains intact, inactivated virus, whereas the subvirion vaccine contains purified virus disrupted with detergents that solubilize the lipid-containing viral envelope, followed by chemical inactivation of residual virus. Attenuated viral vaccines against influenza are also in development.

Currently available methods of detecting influenza infection include virology tests, which involve isolation of the virus from embryonated eggs, commercially available immunodiagnostic tests for influenza antigens such as Binax NOW FluA and FluB™ (Binax, Inc., Portland, Me.), Directigen Flu A+B™ (Becton Dickinson, Franklin Lakes, N.J.), Flu OIA™ (Biostar Inc., Boulder, Colo.), Quick Vue™ (Quidel, Sand Diego, Calif.), Influ AB Quick™ (Denka Sieken Co., Ltd., Japan) and Xpect Flu A & B (Remel Inc., Lenexa, Kans.), or the reverse-transcriptase PCR-based diagnostic test for confirming influenza A virus.

PCT Publication No. WO 03/068201 discloses antibodies having a T-cell receptor-like specificity, yet higher affinity, and the use of same in the detection and treatment of cancer, viral infection and autoimmune disease.

U.S. patent application Ser. Nos. 10/371,942 and 11/582,416 disclose MHC-peptide complex binding ligands.

PCT Publication No. WO 04/084798 discloses antigen-presenting complex-binding compositions and uses thereof.

Additional background art includes U.S. Patent Application No. 20080124272.

### SUMMARY OF THE INVENTION

According to an aspect of some embodiments of the present invention there is provided an isolated antibody comprising an antigen recognition domain which comprises complementarity determining region (CDR) amino acid sequences as set forth in SEQ ID NOs:3-8.

According to an aspect of some embodiments of the present invention there is provided an isolated antibody comprising an antigen recognition domain capable of binding an MHC molecule being complexed with an influenza peptide derived from an influenza polypeptide selected from the group consisting of SEQ ID NOs: 19, 20, 21, 22, 23, 24, 25, 26, 27, 28, 29, 30, 31, 32, 33, 34 and 35, wherein the antibody does not bind the MHC molecule in an absence of the complexed peptide, and wherein the antibody does not bind the peptide in an absence of the MHC molecule.

According to an aspect of some embodiments of the present invention there is provided a molecule comprising the antibody of the invention conjugated to a therapeutic moiety.

According to an aspect of some embodiments of the present invention there is provided a molecule comprising the antibody of the invention conjugated to a detectable moiety.

According to an aspect of some embodiments of the present invention there is provided a multivalent composition comprising the isolated antibody of the invention or the molecule of the invention.

According to an aspect of some embodiments of the present invention there is provided an isolated polynucleotide comprising a nucleic acid sequence encoding the antibody of the invention, or the molecule of the invention.

According to an aspect of some embodiments of the present invention there is provided a nucleic acid construct comprising the isolated polynucleotide of the invention and a promoter for directing expression of the nucleic acid sequence in a host cell.

According to an aspect of some embodiments of the present invention there is provided a pharmaceutical composition comprising as an active ingredient the antibody of the invention, the molecule of the invention, the multivalent composition of the invention, the isolated polynucleotide of the invention and/or the nucleic acid construct of the invention.

According to an aspect of some embodiments of the present invention there is provided a method of detecting a cell expressing an influenza antigen, comprising contacting the cell with the antibody of the invention, the molecule of the invention or the multivalent composition of the invention, under conditions which allow immunocomplex formation, wherein a presence or a level above a predetermined threshold of the immunocomplex is indicative of influenza expression in the cell.

According to an aspect of some embodiments of the present invention there is provided a method of diagnosing an influenza infection in a subject in need thereof, comprising contacting a biological sample of the subject with the antibody of the invention, the molecule of the invention or the multivalent composition of the invention under conditions which allow immunocomplex formation, wherein a presence or a level above a pre-determined threshold of the immunocomplex in the biological sample is indicative of the influenza infected-cells in the subject, thereby diagnosing influenza infection in the subject.

According to an aspect of some embodiments of the present invention there is provided a method of treating an influenza infection, comprising administering to a subject in need thereof a therapeutically effective amount of the antibody of the invention, the molecule of the invention, the multivalent composition of the invention, the isolated polynucleotide of the invention or the nucleic acid construct of the invention, thereby treating the influenza infection.

According to an aspect of some embodiments of the present invention there is provided use of the antibody of the invention, the molecule of the invention or the multivalent composition of the invention, for the manufacture of a medicament for treating influenza infection.

According to an aspect of some embodiments of the present invention there is provided use of the isolated polynucleotide of the invention or the nucleic acid construct of the invention, for the manufacture of a medicament for treating influenza infection.

According to some embodiments of the invention, the antibody of the invention, being multivalent According to some embodiments of the invention, the antibody of the invention, being of an IgG class.

According to some embodiments of the invention, the nucleic acid sequence comprises SEQ ID NOs:9-14.

According to some embodiments of the invention, the antibody of the invention, the antibody, the molecule, the multivalent composition, the isolated polynucleotide and/or said nucleic acid construct is capable of killing influenza-infected cells in the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 depicts ELISA of Fab antibody clones against the HLA-A2/M1₅₈₋₆₆ (SEQ ID NO:2) complex. Reactivity of Fab antibody clones from the screening method with recombinant purified M1/HLA-A2 (M1) or control complex hTERT-540/HLA-A2 (540). ScHLA-A2-peptide complexes were generated by in vitro refolding as described in materials and methods. Detection was with Peroxidase-labeled anti-human Fab. Note the selective binding of Fab antibody clones M1-A2, M1-B3, M1-D1, M1-C5, M1-A10, M1-E6, M1-B11, M1-F11, M1-D9, M1-C8, M1-D12, M1-D3, M1-G3, M1-F8, M1-G8, M1-H3, M1-B12 and M1-E11 to the influenza-M1/HLA-A2 complex but not to the hTERT-540/HLA-A2 complex.

FIG. 2 depicts binding of soluble purified Fab antibodies with TCR-like specificity in ELISA. Binding of soluble Fab antibodies to immobilized MHC-peptide complexes containing various HLA-A2-restricted peptides. Detection was with Peroxidase-labeled anti-human Fab. Note the specific and selective binding of the antibodies A2, D12, D1, E6, F8, F11, B12 and H8 to the influenza-M1/HLA-A2 complex (M1) but not to complexes of hTERT-540/HLA-A2 (540), hTERT-865/HLA-A2 (865) or MART-27/HLA-A2 (Mart), demonstrating that the antibodies are specific to the influenza-M1/HLA-A2 complex and cannot bind the HLA-A2 molecule in the absence of the specific antigen peptide.

FIG. 3 depicts characterization of M1/HLA-A2 specific TCR-like antibodies. Reactivity of four purified Fab antibodies (M1-D12, M1-D1, M1-F8 and M1-G8) with recombinant purified M1/HLA-A2 and control complexes. ScHLA-A2-peptide complexes were generated by in vitro refolding as described under the “General materials and experimental methods”. Detection was with Peroxidase-labeled anti-human Fab. Note the specific binding of the purified Fab antibodies M1-D12, M1-D1, M1-F8 and M1-G8 to the complex of HLA-A2/M1₅₈₋₆₆ but not to complexes of HLA-A2 with the control antigenic peptides MART (SEQ ID NO:38), hTERT-865 (SEQ ID NO:37), hTERT-540 (SEQ ID NO:36), gp100-209 (SEQ ID NO:41), gp100-280 (SEQ ID NO:42), CMV (SEQ ID NO:40), EBV (SEQ ID NO:39) or TAX (SEQ ID NO:44).

FIGS. 4A-L are FACS analyses depicting characterization of the reactivity of purified anti-M1/HLA-A2 TCR-like Fab Abs to peptide-loaded APCs. JY EBV-transformed HLA-A2 positive B cells were loaded with the M1 (SEQ ID NO:2) or control HLA-A2-restricted peptide hTERT-865 (SEQ ID NO:37) and the reactivity with purified Fabs was detected by FACS. FIG. 4A—M1-E6 with JY cells loaded with M1 peptide; FIG. 4B—M1-E6 with JY cells loaded with hTERT-865 peptide; FIG. 4C—M1-D12 with JY cells loaded with M1 peptide; FIG. 4D—M1-D12 with JY cells loaded with hTERT-865 peptide; FIG. 4E—M1-D1 with JY cells loaded with M1 peptide; FIG. 4F—M1-D1 with JY cells loaded with hTERT-865 peptide; FIG. 4G—M1-F8 with JY cells loaded with M1 peptide; FIG. 4H—M1-F8 with JY cells loaded with hTERT-865 peptide; FIG. 4I—M1-G8 with JY cells loaded with M1 peptide; FIG. 4J—M1-G8 with JY cells loaded with hTERT-865 peptide; FIG. 4K—M1-A2 with JY cells loaded with M1 peptide; FIG. 4L—M1-A2 with JY cells loaded with hTERT-865 peptide. Black line=anti human Fab labeled with FITC (the second antibody alone); Red line=M1 Fabs+anti human Fab-FITC.

FIG. 5 is a FACS analysis depicting binding of the TCR-like D12 Fab Ab to peptide-loaded APCs. JY EBV-transformed HLA-A2 positive B cells were loaded with the M1 peptide (SEQ ID NO:2) or with control HLA-A2-restricted peptides [hTERT₈₆₅ (SEQ ID NO:37); hTERT₅₄₀ (SEQ ID NO:36); EBV (SEQ ID NO:39); CMV (SEQ ID NO:40); gp100₂₀₉ (SEQ ID NO:41); gp100₂₈₀ (SEQ ID NO:42); TAX (SEQ ID NO:44); MART-1 (SEQ ID NO:38); gp100₁₅₄ (SEQ ID NO:43); MUC1₁₃₋₂₁ (SEQ ID NO:45)] and the reactivity with the purified D12 Fab was detected by FACS.

FIGS. 6A-B are FACS analyses depicting the binding of D12 Fab tetramer (FIG. 6B) or monomer (FIG. 6A) to peptide-pulsed APCs. JY APCs were pulsed with M1₅₈₋₆₆ (SEQ ID NO:2, red lines) or a control peptide (gp100-209, SEQ ID NO:41, black lines) and were then incubated with the HLA-A2/M1-specific, PE-labeled E5 Fab-tetramer (FIG. 6B) or with the monomer (FIG. 6A). Fab monomer binding was detected with PE-labeled anti-human Fab;

FIGS. 7A-D depict sequences of the D12 antibody which specifically binds the HLA-A2/M1 complex. FIG. 7A—amino acid sequence of the light chain (SEQ ID NO:15); FIG. 7B—nucleic acid sequence of the light chain (SEQ ID NO:16); FIG. 7C—amino acid sequence of the heavy chain (SEQ ID NO:17); FIG. 7D—nucleic acid sequence of the heavy chain (SEQ ID NO:18). CDRs are marked in red; Constant region sequences are highlighted in yellow.

### DESCRIPTION OF SPECIFIC EMBODIMENTS OF THE INVENTION

The present invention, in some embodiments thereof, relates to isolated antibodies which specifically bind a complex of an MHC and an influenza antigen, and more particularly, but not exclusively, to methods of using same for detecting cells infected with the influenza virus and diagnosing and treating influenza infection in a subject.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

While reducing the invention to practice, the present inventors have isolated antibodies which can specifically bind to a complex of an MHC heavy chain and an influenza MHC-restricted antigen but not to the MHC heavy chain or the influenza antigen when not in complex.

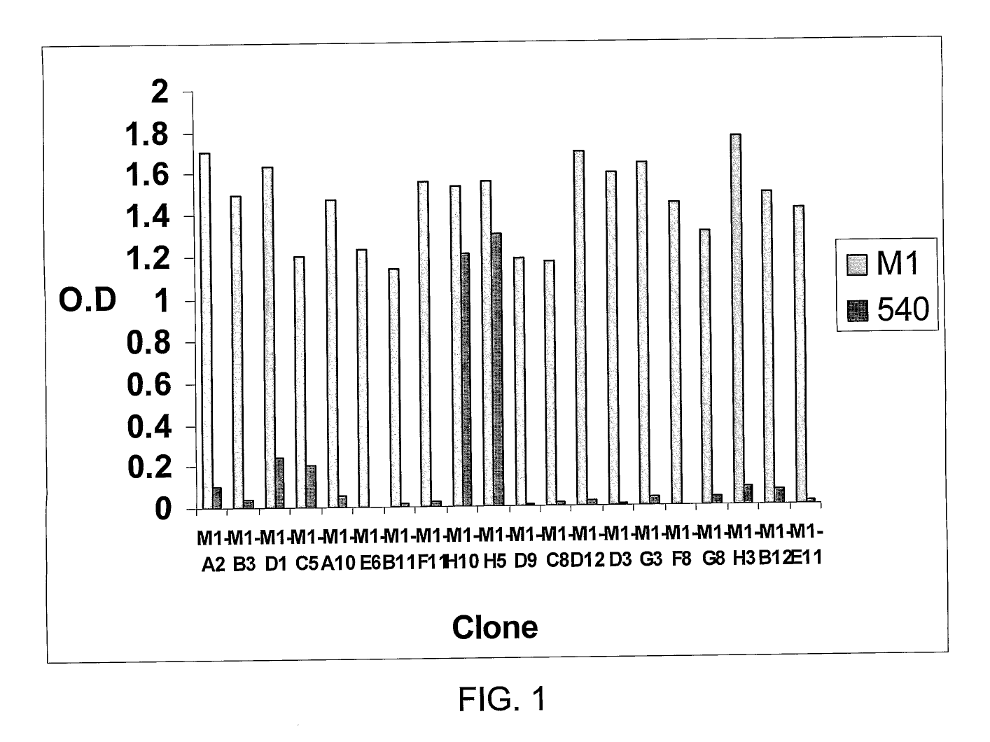

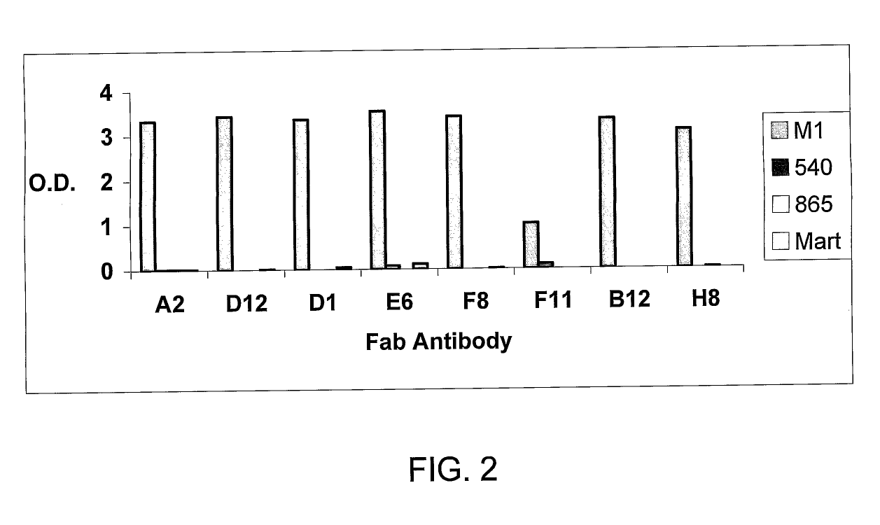

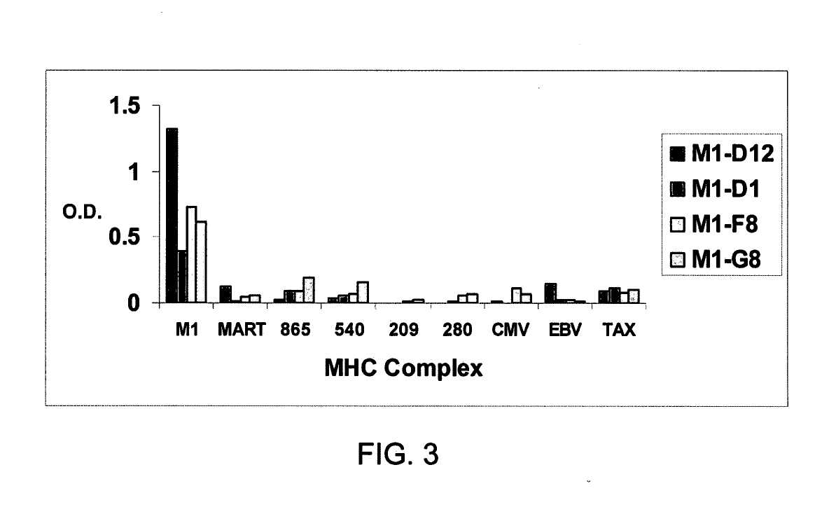

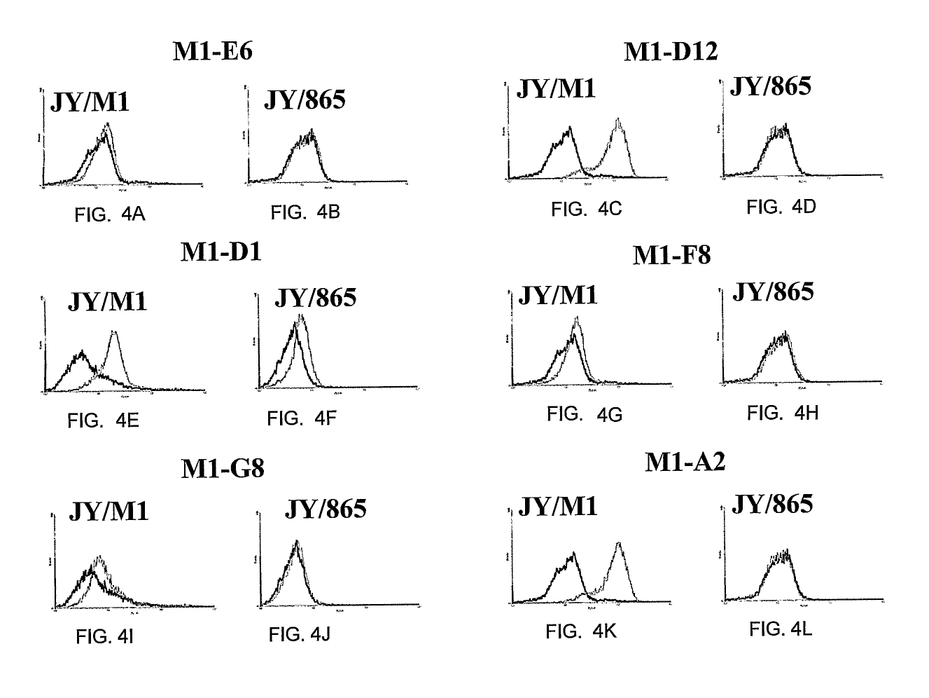

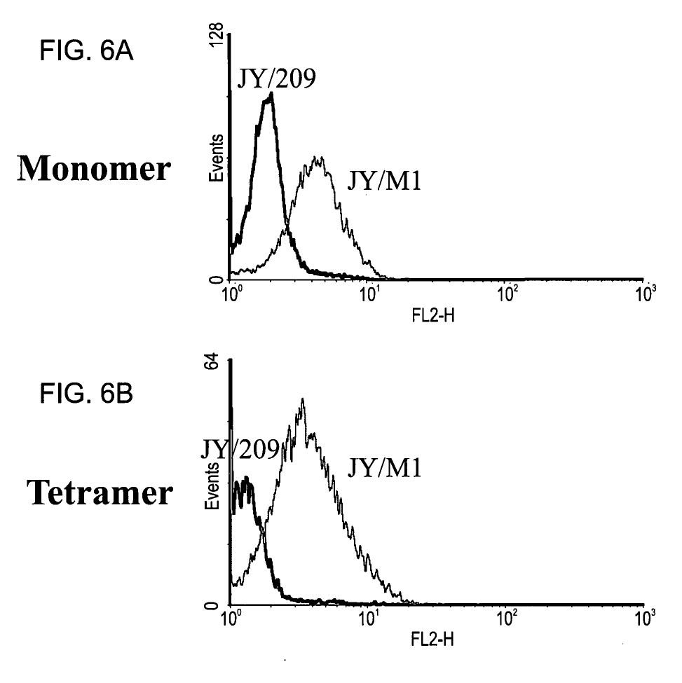

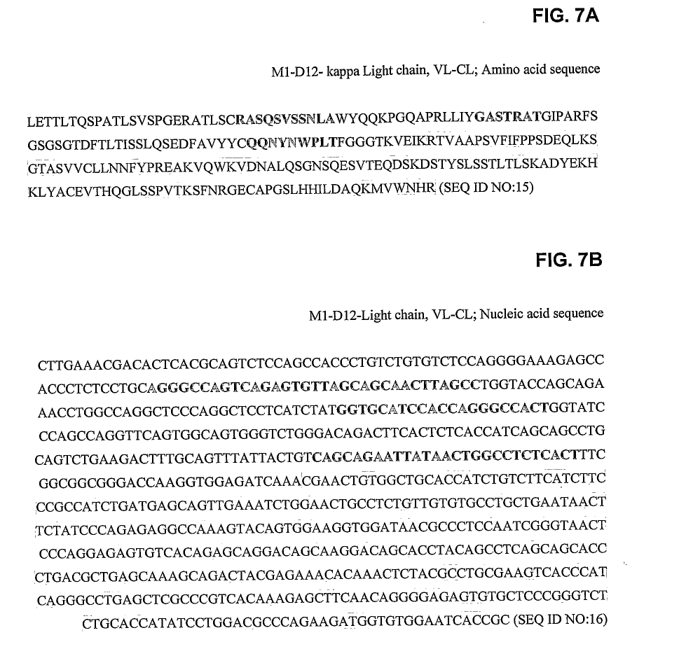

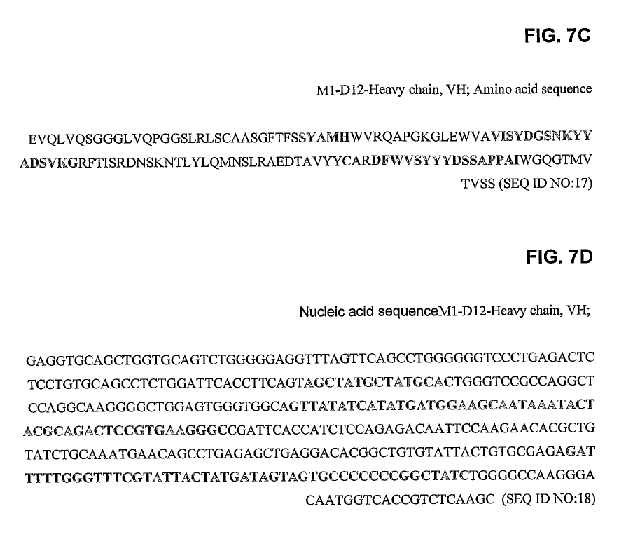

Thus, as described in the Examples section which follows, the present inventors have isolated antibodies (e.g., antibody clones A2, B3, D1, C5, A10, E6, B11, F11, D9, C8, D12, D3, G3, F8, G8, H3, B12, E11, E6, and H8) which specifically bind the complex of HLA-A2/influenza M1₅₈₋₆₆ (SEQ ID NO:2) but not complexes of the HLA-A2 and control antigenic peptides nor to the antigenic peptide alone (FIGS. 1 and 2 and data not shown; Example 1). Further analysis with purified Fab fragments revealed selective binding to the MHC-peptide complex against which the antibodies were selected and not to other MHC-peptide complexes (FIG. 3; Example 1). In addition, the isolated antibodies of the invention were shown capable of binding the specific MHC/M1₅₈₋₆₆ complex presented on the surface of cells (FIGS. 4A-L and 5; Example 2). Moreover, Fab-tetramers generated from the D12 Fab antibody (sequences thereof are shown in FIGS. 7A-D) exhibited increased avidity and specificity to the specific MHC/M1₅₈₋₆₆ complex when displayed on cells (FIGS. 6A-B; Example 3), and thus can be used to detect cells infected with the influenza virus. These highly selective antibodies can be used to diagnose and treat influenza infection in a subject.

Thus, according to an aspect of some embodiments of the present invention there is provided an isolated antibody comprising an antigen recognition domain which binds an MHC molecule being complexed with an influenza peptide derived from an influenza polypeptide, wherein the antibody does not bind the MHC molecule in an absence of the complexed peptide, and wherein the antibody does not bind the peptide in an absence of the MHC molecule.

As used herein the term “isolated” refers to at least partially separated from the natural environment e.g., the human body.

According to some embodiments the term “isolated” refers to a soluble molecule.

According to some embodiments of the invention, the antigen recognition domain of the isolated antibody of the invention comprises complementarity determining region (CDR) amino acid sequences as set forth in SEQ ID NOs:3-8. The light chain of the antibody comprises CDRs amino acid sequences SEQ ID NOs:3 (CDR1), 4 (CDR2) and 5 (CDR3); and the heavy chain of the antibody comprises CDRs amino acid sequences SEQ ID NOs:6 (CDR1), 7 (CDR2) and 8 (CDR3).

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

According to some embodiments of the invention, the antibodies are multivalent forms such as tetrameric Fabs, IgM or IgG1 antibodies, thus forming a multivalent composition with higher avidity to the target. Exemplary methods for generating tetrameric Fabs or IgG1 antibodies are described in the general materials and experimental methods of the Examples section herein below.

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including screening of phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

For in vivo use (for administering in a subject, e.g., human), the human or humanized antibody will generally tend to be better tolerated immunologically than one of non human origin since non variable portions of non human antibodies will tend to trigger xenogeneic immune responses more potent than the allogeneic immune responses triggered by human antibodies which will typically be allogeneic with the individual. It will be preferable to minimize such immune responses since these will tend to shorten the half-life, and hence the effectiveness, of the antibody in the individual. Furthermore, such immune responses may be pathogenic to the individual, for example by triggering harmful inflammatory reactions.

Alternately, an antibody of a human origin, or a humanized antibody, will also be advantageous for applications (such as targeted cell killing) in which a functional physiological effect, for example an immune response against a target cell, activated by a constant region of the antibody in the individual is desired. In these cases, an optimal functional interaction occurs when the functional portion of the antibody, such as the Fc region, and the molecule interacting therewith such as the Fc receptor or the Fc-binding complement component are of a similar origin (e.g., human origin).

Depending on the application and purpose, the antibody of the invention, which includes a constant region, or a portion thereof of any of various isotypes, may be employed. According to some embodiments of the invention, the isotype is selected so as to enable or inhibit a desired physiological effect, or to inhibit an undesired specific binding of the antibody via the constant region or portion thereof. For example, for inducing antibody-dependent cell mediated cytotoxicity (ADCC) by a natural killer (NK) cell, the isotype can be IgG; for inducing ADCC by a mast cell/basophil, the isotype can be IgE; and for inducing ADCC by an eosinophil, the isotype can be IgE or IgA. For inducing a complement cascade the antibody may comprise a constant region or portion thereof capable of initiating the cascade. For example, the antibody may advantageously comprise a Cgamma2 domain of IgG or Cmu3 domain of IgM to trigger a C1q-mediated complement cascade.

Conversely, for avoiding an immune response, such as the aforementioned one, or for avoiding a specific binding via the constant region or portion thereof, the antibody of the invention may not comprise a constant region (be devoid of a constant region), a portion thereof or specific glycosylation moieties (required for complement activation) of the relevant isotype.

As mentioned above, the antibody fragment can be a CDR peptide. Once the CDRs of an antibody are identified, using conventional genetic engineering techniques, expressible polynucleotides encoding any of the forms or fragments of antibodies described herein can be synthesized and modified in one of many ways in order to produce a spectrum of related-products.

For example, to generate the antibody of the invention, an isolated polynucleotide sequence [e.g., SEQ ID NOs:9 (CDR1 of the D12 Ab light chain), 10 (CDR2 of the D12 Ab light chain), 11 (CDR3 of the D12 Ab light chain), 12 (CDR1 of the D12 Ab heavy chain), 13 (CDR2 of the D12 Ab heavy chain), 14 (CDR3 of the D12 Ab heavy chain), 16 (nucleic acid sequence encoding the D12 Ab light chain) or 18 (nucleic acid sequence encoding the D12 Ab heavy chain] encoding the amino acid sequence of the antibody of the invention [e.g., SEQ ID NOs:3 (CDR1 of the D12 Ab light chain), 4 (CDR2 of the D12 Ab light chain), 5 (CDR3 of the D12 Ab light chain), 6 (CDR1 of the D12 Ab heavy chain), 7 (CDR2 of the D12 Ab heavy chain), 8 (CDR3 of the D12 Ab heavy chain), 15 (amino acid sequence of the D12 Ab light chain) or 17 (amino acid sequence of the D12 Ab heavy chain)] is preferably ligated into a nucleic acid construct (expression vector) suitable for expression in a host cell. Such a nucleic acid construct includes a promoter sequence for directing transcription of the polynucleotide sequence in the cell in a constitutive or inducible manner

The nucleic acid construct of the invention may also include an enhancer, a transcription and translation initiation sequence, transcription and translation terminator and a polyadenylation signal, a 5′ LTR, a tRNA binding site, a packaging signal, an origin of second-strand DNA synthesis, and a 3′ LTR or a portion thereof; a signal sequence for secretion of the antibody polypeptide from a host cell; additional polynucleotide sequences that allow, for example, the translation of several proteins from a single mRNA such as an internal ribosome entry site (IRES) and sequences for genomic integration of the promoter-chimeric polypeptide; sequences engineered to enhance stability, production, purification, yield or toxicity of the expressed peptide.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Various methods can be used to introduce the nucleic acid construct of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Recombinant viral vectors are useful for in vivo expression since they offer advantages such as lateral infection and targeting specificity. Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses.

As mentioned hereinabove, a variety of prokaryotic or eukaryotic cells can be used as host-expression systems to express the antibody of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the antibody of the invention.

Recovery of the recombinant antibody polypeptide is effected following an appropriate time in culture. The phrase “recovering the recombinant polypeptide” refers to collecting the whole fermentation medium containing the polypeptide and need not imply additional steps of separation or purification. Not withstanding the above, antibody polypeptides of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

As used herein, the phrase “major histocompatibility complex (MHC)” refers to a complex of antigens encoded by a group of linked loci, which are collectively termed H-2 in the mouse and human leukocyte antigen (HLA) in humans. The two principal classes of the MHC antigens, class I and class II, each comprise a set of cell surface glycoproteins which play a role in determining tissue type and transplant compatibility. In transplantation reactions, cytotoxic T-cells (CTLs) respond mainly against foreign class I glycoproteins, while helper T-cells respond mainly against foreign class II glycoproteins.

MHC class I molecules are expressed on the surface of nearly all cells. These molecules function in presenting peptides which are mainly derived from endogenously synthesized proteins to CD8+ T cells via an interaction with the αβ T-cell receptor. The class I MHC molecule is a heterodimer composed of a 46-kDa heavy chain, which is non-covalently associated with the 12-kDa light chain β-2 microglobulin. In humans, there are several MHC haplotypes, such as, for example, HLA-A2, HLA-A1, HLA-A3, HLA-A24, HLA-A28, HLA-A31, HLA-A33, HLA-A34, HLA-B7, HLA-B45 and HLA-Cw8, their sequences can be found at the kabbat data base [hyper text transfer protocol://immuno (dot) bme (dot) nwu (dot) edu]. Further information concerning MHC haplotypes can be found in Paul, B. Fundamental Immunology Lippincott-Rven Press.

Recombinant soluble MHC class I and class II complexes can be produced in large quantities are described in, for example, Denkberg, G. et al. 2002, and further in U.S. patent application Ser. No. 09/534,966 and PCT/IL01/00260 (published as WO 01/72768), all of which are incorporated herein by reference. Such soluble MHC class I molecules can be loaded with suitable HLA-restricted epitopes and used for vaccination (immunization) of non-human mammal having cells expressing the human MHC class I molecule (see Pascolo et al., J. Exp. Med. 185: 2043-2051, 1997) or be further used for screening antibodies libraries (e.g., the phage display Fab library described in the Examples section which follows).

The influenza MHC-restricted peptide can be derived from any polypeptide produced by the influenza virus. These include, but not limited to membrane protein M1 [e.g., of influenza A virus A/Korea/426/68(H2N2) GenBank Accession No. YP_{—}308854.1; SEQ ID NO:1]; hemagglutinin [3 distinct hemagglutinins, H1, H2, and H3 are found in human infections; e.g., hemagglutinin of influenza B virus (GenBank Accession No. NP_{—}056660.1; SEQ ID NO:32); hemagglutinin of influenza A virus (A/New York/392/2004(H3N2) GenBank Accession No. YP_{—}308839.1; SEQ ID NO:35]; neuraminidase (NA) [2 different neuraminidases N1 and N2 have been found in human viruses; e.g., neuraminidase of influenza B virus GenBank Accession No. NP_{—}056663.1; SEQ ID NO:30]; the nucleoprotein (NP) [Influenza A, B, and C viruses have different nucleoproteins; e.g., nucleoprotein of influenza C virus (GenBank Accession No. YP_{—}089656.1; SEQ ID NO:29), nucleoprotein of influenza A virus such as the A/Korea/426/68(H2N2) strain GenBank Accession No. YP_{—}308871.1, SEQ ID NO:31 or the A/Hong Kong/1073/99(H9N2) strain GenBank Accession No. YP_{—}581749.1; SEQ ID NO:34], nucleoprotein of influenza B virus (GenBank Accession No. NP_{—}056661.1; SEQ ID NO:33)]; matrix protein (M1) [e.g., of influenza B virus GenBank Accession No. NP_{—}056664.1; SEQ ID NO:21]; the ion channel (M2) [e.g., of influenza A virus A/Puerto Rico/8/34(H1N1) strain GenBank Accession No. NP_{—}040979.2; SEQ ID NO:19]; non-structural protein NS-1 [e.g., of influenza B virus GenBank Accession No. NP_{—}056666.1; SEQ ID NO:23]; non-structural protein NS-2 [e.g., of influenza B virus GenBank Accession No. NP_{—}056665.1; SEQ ID NO:26]; PA [e.g., of influenza A virus A/Charlottesville/28/95(H1N1), GenBank Accession No. AAL60433; SEQ ID NO:20]; PB1 [e.g., of influenza B virus GenBank Accession No. NP_{—}056657.1; SEQ ID NO:22]; PB2 [e.g., of influenza A virus (A/Puerto Rico/8/34(H1N1), GenBank Accession No. NP_{—}040987.1; SEQ ID NO:24]; BM2 protein [e.g., of influenza B virus GenBank Accession No. YP_{—}419283.1; SEQ ID NO:25]; NB protein [e.g., of influenza B virus GenBank Accession No. NP_{—}056662.1; SEQ ID NO:27]; or the nucleocapsid protein [e.g., of influenza A virus A/Puerto Rico/8/34(H1N1); GenBank Accession No. NP_{—}040982.1; SEQ ID NO:28]. Additional sequences of the influenza polypeptides are available through the National Center for Biotechnology Information [Hypertext Transfer Protocol://World Wide Web (dot) ncbi (dot) nlm (dot) nih (dot) gov/].

According to some embodiments of the invention, the influenza polypeptide from which the influenza peptide is derived is selected from the group consisting of SEQ ID NOs: 19, 20, 21, 22, 23, 24, 25, 26, 27, 28, 29, 30, 31, 32, 33, 34 and 35.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time. According to some embodiments of the invention, but not in all cases necessary, these modifications should exclude anchor amino acids.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

The peptides of the invention are preferably utilized in a linear form, although it will be appreciated that in cases where cyclicization does not severely interfere with peptide characteristics, cyclic forms of the peptide can also be utilized.

The peptides of the invention may include one or more non-natural or natural polar amino acids, including but not limited to serine and threonine which are capable of increasing peptide solubility due to their hydroxyl-containing side chain.

The peptides of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965. Large scale peptide synthesis is described by Andersson Biopolymers 2000; 55(3):227-50.

Based on accumulated experimental data, it is nowadays possible to predict which of the peptides of a protein will bind to MHC class I. The HLA-A2 MHC class I has been so far characterized better than other HLA haplotypes, yet predictive and/or sporadic data is available for all other haplotypes.

With respect to HLA-A2 binding peptides, assume the following positions (P1-P9) in a 9-mer peptide: P1-P2-P3-P4-P5-P6-P7-P8-P9.

The P2 and P2 positions include the anchor residues which are the main residues participating in binding to MHC molecules. Amino acid resides engaging positions P2 and P9 are hydrophilic aliphatic non-charged natural amino (examples being Ala, Val, Leu, Ile, Gln, Thr, Ser, Cys, preferably Val and Leu) or of a non-natural hydrophilic aliphatic non-charged amino acid [examples being norleucine (Nle), norvaline (Nva), α-aminobutyric acid]. Positions P1 and P3 are also known to include amino acid residues which participate or assist in binding to MHC molecules, however, these positions can include any amino acids, natural or non-natural. The other positions are engaged by amino acid residues which typically do not participate in binding, rather these amino acids are presented to the immune cells. Further details relating to the binding of peptides to MHC molecules can be found in Parker, K. C., Bednarek, M. A., Coligan, J. E., Scheme for ranking potential HLA-A2 binding peptides based on independent binding of individual peptide side-chains. J Immunol. 152, 163-175, 1994, see Table V, in particular. Hence, scoring of HLA-A2.1 binding peptides can be performed using the HLA Peptide Binding Predictions software approachable through a worldwide web interface at hypertexttransferprotocol://worldwideweb (dot) bimas (dot) dcrt (dot) nih (dot) gov/molbio/hla_bind/index. This software is based on accumulated data and scores every possible peptide in an analyzed protein for possible binding to MHC HLA-A2.1 according to the contribution of every amino acid in the peptide. Theoretical binding scores represent calculated half-life of the HLA-A2.1-peptide complex.

Hydrophilic aliphatic natural amino acids at P2 and P9 can be substituted by synthetic amino acids, preferably Nleu, Nval and/or α-aminobutyric acid. P9 can be also substituted by aliphatic amino acids of the general formula —HN(CH2)nCOOH, wherein n=3-5, as well as by branched derivatives thereof, such as, but not limited to,

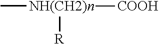

wherein R is, for example, methyl, ethyl or propyl, located at any one or more of the n carbons.

The amino terminal residue (position P1) can be substituted by positively charged aliphatic carboxylic acids, such as, but not limited to, H2N(CH2)nCOOH, wherein n=2-4 and H2N—C(NH)—NH(CH2)nCOOH, wherein n=2-3, as well as by hydroxy Lysine, N-methyl Lysine or ornithine (Orn). Additionally, the amino terminal residue can be substituted by enlarged aromatic residues, such as, but not limited to, H2N—(C6H6)-CH2-COOH, p-aminophenyl alanine, H2N—F(NH)—NH—(C6H6)-CH2-COOH, p-guanidinophenyl alanine or pyridinoalanine (Pal). These latter residues may form hydrogen bonding with the OH— moieties of the influenza residues at the MHC-1 N-terminal binding pocket, as well as to create, at the same time aromatic-aromatic interactions.

Derivatization of amino acid residues at positions P4-P8, should these residues have a side-chain, such as, OH, SH or NH2, like Ser, Tyr, Lys, Cys or Orn, can be by alkyl, aryl, alkanoyl or aroyl. In addition, OH groups at these positions may also be derivatized by phosphorylation and/or glycosylation. These derivatizations have been shown in some cases to enhance the binding to the T cell receptor.

Longer derivatives in which the second anchor amino acid is at position P10 may include at P9 most L amino acids. In some cases shorter derivatives are also applicable, in which the C terminal acid serves as the second anchor residue.

Cyclic amino acid derivatives can engage position P4-P8, preferably positions P6 and P7. Cyclization can be obtained through amide bond formation, e.g., by incorporating Glu, Asp, Lys, Orn, di-amino butyric (Dab) acid, di-aminopropionic (Dap) acid at various positions in the chain (—CO—NH or —NH—CO bonds). Backbone to backbone cyclization can also be obtained through incorporation of modified amino acids of the formulas H—N((CH2)n-COOH)—C(R)H—COOH or H—N((CH2)n-COOH)—C(R)H—NH2, wherein n=1-4, and further wherein R is any natural or non-natural side chain of an amino acid.

Cyclization via formation of S—S bonds through incorporation of two Cys residues is also possible. Additional side-chain to side chain cyclization can be obtained via formation of an interaction bond of the formula —(—CH2-)n-S—CH2-C—, wherein n=1 or 2, which is possible, for example, through incorporation of Cys or homoCys and reaction of its free SH group with, e.g., bromoacetylated Lys, Orn, Dab or Dap.

According to an aspect of some embodiments of the invention, there is provided a molecule comprising the antibody of the invention being conjugated to a functional moiety (also referred to as an “immunoconjugate”) such as a detectable or a therapeutic moiety. The immunoconjugate molecule can be an isolated molecule such as a soluble or synthetic molecule.

Various types of detectable or reporter moieties may be conjugated to the antibody of the invention. These include, but not are limited to, a radioactive isotope (such as ^{[125]}iodine), a phosphorescent chemical, a chemiluminescent chemical, a fluorescent chemical (fluorophore), an enzyme, a fluorescent polypeptide, an affinity tag, and molecules (contrast agents) detectable by Positron Emission Tomagraphy (PET) or Magnetic Resonance Imaging (MRI).

Examples of suitable fluorophores include, but are not limited to, phycoerythrin (PE), fluorescein isothiocyanate (FITC), Cy-chrome, rhodamine, green fluorescent protein (GFP), blue fluorescent protein (BFP), Texas red, PE-Cy5, and the like. For additional guidance regarding fluorophore selection, methods of linking fluorophores to various types of molecules see Richard P. Haugland, “Molecular Probes: Handbook of Fluorescent Probes and Research Chemicals 1992-1994”, 5th ed., Molecular Probes, Inc. (1994); U.S. Pat. No. 6,037,137 to Oncoimmunin Inc.; Hermanson, “Bioconjugate Techniques”, Academic Press New York, N.Y. (1995); Kay M. et al., 1995. Biochemistry 34:293; Stubbs et al., 1996. Biochemistry 35:937; Gakamsky D. et al., “Evaluating Receptor Stoichiometry by Fluorescence Resonance Energy Transfer,” in “Receptors: A Practical Approach,” 2nd ed., Stanford C. and Horton R. (eds.), Oxford University Press, UK. (2001); U.S. Pat. No. 6,350,466 to Targesome, Inc.]. Fluorescence detection methods which can be used to detect the antibody when conjugated to a fluorescent detectable moiety include, for example, fluorescence activated flow cytometry (FACS), immunofluorescence confocal microscopy, fluorescence in-situ hybridization (FISH) and fluorescence resonance energy transfer (FRET).

Numerous types of enzymes may be attached to the antibody of the invention [e.g., horseradish peroxidase (HPR), beta-galactosidase, and alkaline phosphatase (AP)] and detection of enzyme-conjugated antibodies can be performed using ELISA (e.g., in solution), enzyme-linked immunohistochemical assay (e.g., in a fixed tissue), enzyme-linked chemiluminescence assay (e.g., in an electrophoretically separated protein mixture) or other methods known in the art [see e.g., Khatkhatay M I. and Desai M., 1999. J Immunoassay 20:151-83; Wisdom G B., 1994. Methods Mol Biol. 32:433-40; Ishikawa E. et al., 1983. J Immunoassay 4:209-327; Oellerich M., 1980. J Clin Chem Clin Biochem. 18:197-208; Schuurs A H. and van Weemen B K., 1980. J Immunoassay 1:229-49).

The affinity tag (or a member of a binding pair) can be an antigen identifiable by a corresponding antibody [e.g., digoxigenin (DIG) which is identified by an anti-DIG antibody) or a molecule having a high affinity towards the tag [e.g., streptavidin and biotin]. The antibody or the molecule which binds the affinity tag can be fluorescently labeled or conjugated to enzyme as described above.

Various methods, widely practiced in the art, may be employed to attach a streptavidin or biotin molecule to the antibody of the invention. For example, a biotin molecule may be attached to the antibody of the invention via the recognition sequence of a biotin protein ligase (e.g., BirA) as described in the Examples section which follows and in Denkberg, G. et al., 2000. Eur. J. Immunol. 30:3522-3532. Alternatively, a streptavidin molecule may be attached to an antibody fragment, such as a single chain Fv, essentially as described in Cloutier S M. et al., 2000. Molecular Immunology 37:1067-1077; Dubel S. et al., 1995. J Immunol Methods 178:201; Huston J S. et al., 1991. Methods in Enzymology 203:46; Kipriyanov S M. et al., 1995. Hum Antibodies Hybridomas 6:93; Kipriyanov S M. et al., 1996. Protein Engineering 9:203; Pearce L A. et al., 1997. Biochem Molec Biol Intl 42:1179-1188).

Functional moieties, such as fluorophores, conjugated to streptavidin are commercially available from essentially all major suppliers of immunofluorescence flow cytometry reagents (for example, Pharmingen or Becton-Dickinson).

According to some embodiments of the invention, biotin conjugated antibodies are bound to a streptavidin molecule to form a multivalent composition (e.g., a dimmer or tetramer form of the antibody).

Table 1 provides non-limiting examples of identifiable moieties which can be conjugated to the antibody of the invention.

TABLE 1

| | Amino Acid sequence | Nucleic Acid sequence |
|---|---|---|
| | (GenBank Accession No.)/ | (GenBank Accession No.)/ |
| Identifiable Moiety | SEQ ID NO: | SEQ ID NO: |
| Green Fluorescent protein | AAL33912/49 | AF435427/50 |
| Alkaline phosphatase | AAK73766/51 | AY042185/52 |
| Peroxidase | CAA00083/53 | A00740/54 |
| Histidine tag | Amino acids 264-269 of | Nucleotides 790-807 of |
| | GenBank Accession No. | GenBank Accession No. |
| | AAK09208/55 | AF329457/56 |
| Myc tag | Amino acids 273-283 of | Nucleotides 817-849 of |
| | GenBank Accession No. | GenBank Accession No. |
| | AAK09208/57 | AF329457/58 |
| Biotin lygase tag | LHHILDAQKMVWNHR/46 | |
| orange fluorescent protein | AAL33917/59 | AF435432/60 |
| Beta galactosidase | ACH42114/61 | EU626139/62 |
| Streptavidin | AAM49066/63 | AF283893/64 |

Table 1.

As mentioned, the antibody may be conjugated to a therapeutic moiety. The therapeutic moiety can be, for example, a cytotoxic moiety, a toxic moiety, a cytokine moiety and a second antibody moiety comprising a different specificity to the antibodies of the invention.

Non-limiting examples of therapeutic moieties which can be conjugated to the antibody of the invention are provided in Table 2, hereinbelow.

TABLE 2

| | Amino acid sequence | Nucleic acid sequence |
|---|---|---|
| | (GenBank Accession | (GenBank Accession |
| Therapeutic moiety | No.)/SEQ ID NO: | No.)/SEQ ID NO: |
| Pseudomonas exotoxin | ABU63124/65 | EU090068/66 |
| Diphtheria toxin | AAV70486/67 | AY820132.1/68 |
| interleukin 2 | CAA00227/69 | A02159/70 |
| CD3 | P07766/71 | X03884/72 |
| CD16 | NP_000560.5/73 | NM_000569.6/74 |
| interleukin 4 | NP_000580.1/75 | NM_000589.2/76 |
| HLA-A2 | P01892/77 | K02883/78 |
| interleukin 10 | P22301/79 | M57627/80 |
| Ricin toxin | EEF27734/81 | EQ975183/82 |

According to some embodiments of the invention, the toxic moiety is PE38KDEL (SEQ ID NO:83 for the amino acid sequence; SEQ ID NO:84 for the nucleic acid sequence).

The functional moiety (the detectable or therapeutic moiety of the invention) may be attached or conjugated to the antibody of the invention in various ways, depending on the context, application and purpose.

When the functional moiety is a polypeptide, the immunoconjugate may be produced by recombinant means. For example, the nucleic acid sequence encoding a toxin (e.g., PE38KDEL) or a fluorescent protein [e.g., green fluorescent protein (GFP), red fluorescent protein (RFP) or yellow fluorescent protein (YFP)] may be ligated in-frame with the nucleic acid sequence encoding the antibody of the invention (e.g., SEQ ID NOs:16 and 18) and be expressed in a host cell to produce a recombinant conjugated antibody. Alternatively, the functional moiety may be chemically synthesized by, for example, the stepwise addition of one or more amino acid residues in defined order such as solid phase peptide synthetic techniques.

A functional moiety may also be attached to the antibody of the invention using standard chemical synthesis techniques widely practiced in the art [see e.g., hypertexttransferprotocol://worldwideweb (dot) chemistry (dot) org/portal/Chemistry)], such as using any suitable chemical linkage, direct or indirect, as via a peptide bond (when the functional moiety is a polypeptide), or via covalent bonding to an intervening linker element, such as a linker peptide or other chemical moiety, such as an organic polymer. Chimeric peptides may be linked via bonding at the carboxy (C) or amino (N) termini of the peptides, or via bonding to internal chemical groups such as straight, branched or cyclic side chains, internal carbon or nitrogen atoms, and the like. Description of fluorescent labeling of antibodies is provided in details in U.S. Pat. Nos. 3,940,475, 4,289,747, and 4,376,110.

Exemplary methods for conjugating peptide moieties (therapeutic or detectable moieties) to the antibody of the invention are described herein below:

SPDP conjugation—A non-limiting example of a method of SPDP conjugation is described in Cumber et al. (1985, Methods of Enzymology 112: 207-224). Briefly, a peptide, such as a detectable or therapeutic moiety (e.g., 1.7 mg/ml) is mixed with a 10-fold excess of SPDP (50 mM in ethanol); the antibody is mixed with a 25-fold excess of SPDP in 20 mM sodium phosphate, 0.10 M NaCl pH 7.2 and each of the reactions is incubated for about 3 hours at room temperature. The reactions are then dialyzed against PBS. The peptide is reduced, e.g., with 50 mM DTT for 1 hour at room temperature. The reduced peptide is desalted by equilibration on G-25 column (up to 5% sample/column volume) with 50 mM KH₂PO₄ pH 6.5. The reduced peptide is combined with the SPDP-antibody in a molar ratio of 1:10 antibody:peptide and incubated at 4° C. overnight to form a peptide-antibody conjugate.

Glutaraldehyde conjugation—A non-limiting example of a method of glutaraldehyde conjugation is described in G. T. Hermanson (1996, “Antibody Modification and Conjugation, in Bioconjugate Techniques, Academic Press, San Diego). Briefly, the antibody and the peptide (1.1 mg/ml) are mixed at a 10-fold excess with 0.05% glutaraldehyde in 0.1 M phosphate, 0.15 M NaCl pH 6.8, and allowed to react for 2 hours at room temperature. 0.01 M lysine can be added to block excess sites. After-the reaction, the excess glutaraldehyde is removed using a G-25 column equilibrated with PBS (10% v/v sample/column volumes)

Carbodiimide conjugation—Conjugation of a peptide with an antibody can be accomplished using a dehydrating agent such as a carbodiimide, e.g., in the presence of 4-dimethyl aminopyridine. Carbodiimide conjugation can be used to form a covalent bond between a carboxyl group of peptide and an hydroxyl group of an antibody (resulting in the formation of an ester bond), or an amino group of an antibody (resulting in the formation of an amide bond) or a sulfhydryl group of an antibody (resulting in the formation of a thioester bond). Likewise, carbodiimide coupling can be used to form analogous covalent bonds between a carbon group of an antibody and an hydroxyl, amino or sulfhydryl group of the peptide [see, J. March, Advanced Organic Chemistry: Reaction's, Mechanism, and Structure, pp. 349-50 & 372-74 (3d ed.), 1985]. For example, the peptide can be conjugated to an antibody via a covalent bond using a carbodiimide, such as dicyclohexylcarbodiimide [B. Neises et al. (1978), Angew Chem., Int. Ed. Engl. 17:522; A. Hassner et al. (1978, Tetrahedron Lett. 4475); E. P. Boden et al. (1986, J. Org. Chem. 50:2394) and L. J. Mathias (1979, Synthesis 561)].

As mentioned above and further illustrated in the Examples section which follows, the isolated antibodies of the invention can be used to detect the complex of MHC and influenza antigenic peptide on the surface of cells such as influenza-virus infected cells.

Thus, according to an aspect of some embodiments of the invention, there is provided a method of detecting a cell expressing an influenza antigen, comprising contacting the cell with the isolated antibody of the invention, the molecule comprising the antibody conjugated to a detectable moiety and/or the multivalent composition comprising same, under conditions which allow immunocomplex formation, wherein a presence or a level above a predetermined threshold of the immunocomplex is indicative of influenza expression in the cell.

The cell expressing the influenza antigen can be any nucleated cell such as antigen presenting cells (APC) in the blood.

Contacting the cell with the antibody/molecule or multivalent composition of the invention may be effected in vitro (e.g., in a cell line), ex vivo or in vivo.

As mentioned, the method of the invention is effected under conditions sufficient to form an immunocomplex; such conditions (e.g., appropriate concentrations, buffers, temperatures, reaction times) as well as methods to optimize such conditions are known to those skilled in the art, and examples are disclosed herein. As used herein the phrase “immunocomplex” refers to a complex which comprises the antibody of the invention and the MHC-influenza peptide complex. Determining a presence or level of the immunocomplex of the invention is dependent on the detectable moiety to which the antibody is attached, and can be performed using various methods are known in the art and described hereinabove.

The level of the immunocomplex in the tested cell (e.g., a cell of a subject in need thereof) is compared to a predetermined threshold. The threshold may be determined based on a known reference level and/or a level in a control cell. The control cell can be obtained from a control, healthy subject (e.g., a subject not infected with the influenza virus) or from a subject devoid of the specific MHC molecule forming the MHC-peptide complex (e.g., HLA-A2). According to some embodiments of the invention, the control subject is of the same species e.g. human, preferably matched with the same age, weight, sex etc. as the subject in need thereof.

Thus, the teachings of the invention can be used to diagnose an influenza infection in a subject by detecting an influenza-infected cell(s) in a biological sample of the subject.

As used herein the phrase “influenza-infected cell” refers to any cell or a portion thereof of the subject which displays the complex of MHC and MHC-restricted influenza antigen.

The biological sample can be any sample which contains cells or a portion thereof (e.g., cell debris, membrane vesicles) which putatively present the MHC-influenza antigenic peptide complex.

According to some embodiments of the invention, the subject is at risk of infection with the influenza virus, and/or at risk of developing clinical complication therefrom.

As used herein the term “diagnosing” refers to determining presence or absence of a pathology, classifying a pathology or a symptom, determining a severity of the pathology, monitoring pathology progression, forecasting an outcome of a pathology and/or prospects of recovery.

To facilitate diagnosis, the above teachings can be combined with other methods of diagnosing influenza which are well known in the art including but are not limited to clinical symptoms of influenza, various virology tests (e.g., isolation of the virus from embryonated eggs), known immunodiagnostic tests such as Binax NOW FluA and FluB™ (Binax, Inc., Portland, Me.), Directigen Flu A+B™ (Becton Dickinson, Franklin Lakes, N.J.), Flu OIA™ (Biostar Inc., Boulder, Colo.), Quick Vue™ (Quidel, Sand Diego, Calif.), Influ AB Quick™ (Denka Sieken Co., Ltd., Japan) and Xpect Flu A & B (Remel Inc., Lenexa, Kans.), or the reverse-transcriptase PCR-based diagnostic test for confirming influenza A virus.

The teachings of the invention can be used to treat a subject who is infected with the influenza virus.

Thus, according to an aspect of some embodiments of the invention, there is provided a method of treating an influenza infection, comprising administering to a subject in need thereof a therapeutically effective amount of the isolated antibody of the invention, the molecule of the invention (e.g., which includes the antibody conjugated to a therapeutic moiety such as toxin), the multivalent composition comprising same, the isolated polynucleotide or the nucleic acid construct encoding same, thereby treating the influenza infection.

The term “treating” refers to inhibiting or arresting the development of a disease, disorder or condition and/or causing the reduction, remission, or regression of a disease, disorder or condition. Those of skill in the art will understand that various methodologies and assays can be used to assess the development of a disease, disorder or condition, and similarly, various methodologies and assays may be used to assess the reduction, remission or regression of a disease, disorder or condition.

According to some embodiments of the invention, the isolated antibody, molecule, multivalent composition, polynucleotide, and/or nucleic acid construct of the invention is capable of killing influenza-infected cells in the subject in need thereof.

The antibodies of the invention, the molecule of the invention (which comprise the antibody conjugated to a therapeutic or detectable moiety), the multivalent composition of the invention, the isolated polynucleotide or the nucleic acid construct of the invention may be provided per se or may be administered as a pharmaceutical composition.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the antibody of the invention, the molecule of the invention (which comprise the antibody conjugated to a therapeutic or detectable moiety), the multivalent composition of the invention, the isolated polynucleotide or the nucleic acid construct of the invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the invention may also be formulated in rectal compositions such as suppositories or retention enemas, using, e.g., conventional suppository bases such as cocoa butter or other glycerides.

Pharmaceutical compositions suitable for use in context of the invention include compositions wherein the active ingredients are contained in an amount effective to achieve the intended purpose. More specifically, a therapeutically effective amount means an amount of active ingredients [the antibody of the invention, the molecule of the invention (which comprise the antibody conjugated to a therapeutic or detectable moiety), the multivalent composition of the invention, the isolated polynucleotide or the nucleic acid construct of the invention] effective to prevent, alleviate or ameliorate symptoms of a disorder (influenza infection) or prolong the survival of the subject being treated.

Determination of a therapeutically effective amount is well within the capability of those skilled in the art, especially in light of the detailed disclosure provided herein.

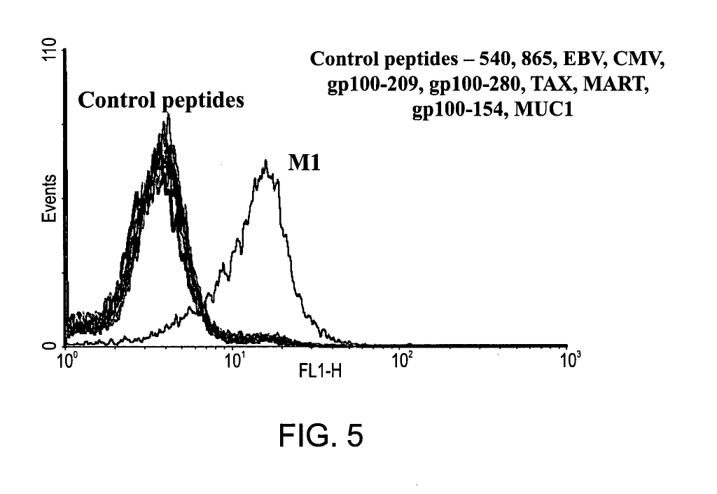

For example, the effect of the active ingredients (e.g., the antibody of the invention) on influenza treatment can be evaluated by monitoring the killing of influenza-infected cells since the antibody binds to class I influenza-derived MHC-peptide complexes presented on influenza-infected cells. Methods of detecting cell killing are known in the art and include, for example, assays which detect protein synthesis (e.g., incorporation of ³H-Leucine into cellular proteins as shown in FIG. 5B), Ethidium homodimer-1 staining (Invitrogen-Molecular Probes), the Tunnel assay (Roche, Basel, Switzerland), the Live/dead viability/cytotoxicity two-color fluorescence assay (Molecular Probes, Inc., L-3224, Eugene, Oreg., USA), FACS analysis [using molecules capable of specifically binding cells undergoing apoptosis, such as propidium iodide and Annexin V] and the like.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

According to some embodiments of the invention, the therapeutic agent of the invention (e.g., the antibody, molecule and/or multivalent composition of the invention) can be provided to the subject in combination with other drug(s) designed for treating influenza (combination therapy). Such a combination therapy may increase the therapeutic effect of the agent of the invention in the treated subject.

Non-limiting examples of known anti-influenza drugs which can be co-administered to the subject along with the therapeutic agent of the invention include, but are not limited to M2 inhibitors (adamantane derivatives) against influenza A such as Amantadine [SYMMETREL (Endo Pharmaceuticals)], and Rimantadine [FLUMADINE (Forest Laboratories)]; neuraminidase inhibitors against influenza A and B such as Zanamivir [RELENZA (GlaxoSmithKline)] and Oseltamivir [TAMIFLU (Hoffmann-La Roche)]; analgesic drugs (e.g., paracetamol); non-steroidal anti-inflammatory drugs (NSAIDs, e.g., salicylates); narcotic drugs (e.g., morphine) or synthetic drugs with narcotic properties (e.g., tramadol); decongestants (e.g., pseudoephedrine, phenylephrine, oxymetazoline; antihistamines; and cough suppressants (antitussives).

Compositions of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

As used herein the term “about” refers to ±10%

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, an and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements. Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes Cellis, J. E., ed. (1994); “Current Protocols in Immunology” Volumes Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

Production of biotinylated scMHC/peptide complexes—To construct a single-chain (sc) major histocompatibility (MHC) BirA (scMHC-BirA) plasmid, a peptide sequence for site specific biotinylation [LHHILDAQKMVWNHR (SEQ ID NO:46), the lysine residue undergoing biotinylation by the BirA biotin ligase enzyme is bolded] was fused at the C-terminus of the HLA-A2. This construct was subcloned into a pET-based expression vector for efficient expression in E. Coli.

Folding and purification of recombinant MHC/peptide complexes or recombinant fusion molecule—Dithioerithriol was added to a final concentration of 65 mM (10 mg/ml) to the solubilized inclusion bodies of scMHC, or fusion molecule (scMHC-BirA molecule) which were incubated for more than 2 hours. The reduced inclusion bodies were diluted 1:100 with refolding buffer (0.1 M Tris-HCl pH=8, 0.5 M Arginine, 0.09 mM oxidized glutathione, 2 mM EDTA, 0.2 mM PMSF) and 5 or 10 fold molar excess of peptide (usually 1 mg/100 ml refolding buffer) was added to scMHC previously diluted in H₂O or DMSO, and incubated at 4-10° C. for 48 hours.

After refolding, the protein was dialyzed against 100 mM Urea, 20 mM Tris-HCl pH=8, and concentrated by a Minisette system using a 10 K cutoff cassette to a final volume of 200 ml. The protein was loaded on Q Sepharose anion exchange column. The column was washed with buffer A containing 5 mM NaCl, 20 mM Tris HCl pH=8, 1 mM EDTA. Relevant fractions corresponding to correctly folded MHC/peptide or fusion molecule monomers were poured to a centricon device (30 kDa cut off) (Amicon, Beverly Mass.) and concentrated to volume 0.3-1.0 ml (usually no more than 2 mg/ml to avoid protein aggregation). The clean fractions were frozen at −70° C. at this step, until further use.

Biotinylation of MHC/peptide complexes—The buffer was exchanged (using the centricon) with 10 mM Tris-HCl, pH=8, 50 mM NaCl. The final protein concentration was brought to 1-2 mg/ml (25-50 μM). Enzymatic biotinylation was performed at a specific lysine residue in the heavy chain C-terminal tag using biotin protein ligase—Bir A enzyme (AVIDITY, Denver, Colo.) for 16 hr at 25° C., in presence of protease inhibitors cocktail (0.1 mM PMSF, 1 μg/ml Leupeptin, 1 μg/ml Pepstatin). The buffer was exchanged and the excess biotin was removed from the biotinylated complexes using centricon 30 ultrafiltration or G-25. The MHC/peptide biotinylated monomers were frozen at −70° C.

Selection of phage-antibodies on biotinylated complexes—A large human Fab library containing 3.7×10¹⁰ different Fab clones was used for the selection (de Haard, H. J. et al. 1999). Phage (10¹³) was first preincubated for 1 hour at room temperature in PBS containing 2% nonfat dry milk with streptavidin-coated paramagnetic beads (200 ml; Dynal, Oslo) to deplete streptavidin binders. Streptavidin-coated paramagnetic beads (200 ml; Dynal, Oslo) were also incubated in phosphate buffer saline (PBS) supplemented with 2% milk for 1 hour at room temperature. The remaining phages were subsequently incubated for 1 hour with decreasing amounts of biotinylated scMHC-peptide complexes. Streptavidin magnetic beads were added, and the mixture was incubated for 15 minutes with continuous rotation. A magnetic force was applied to pull down phages bound to biotinylated complexes. After 10 washes of the streptavidin-bound complexes with PBS containing 0.1% Tween and 2 washes with PBS, bound phages were eluted by incubation for 7 minutes with 1 ml of Triethylamine (TEA) (100 mM). The elusion mixture was neutralized by the addition of 100 μl of Tris-HCl (1 M, pH 7.4) and used to infect E. coli TG1 cells (OD₆₀₀=0.5) for 30 minutes at 37° C.

Selected phages were rescued using M13KO7 helper phage (5×10¹¹ cfu). The diversity of the selected antibodies was determined by DNA fingerprinting. The Fab DNA of different clones was PCR-amplified using the primers pUC-reverse (5′-AGCGGATAACAATTTCACACAGG-3; SEQ ID NO:47) and fd-tet-seq24 (5′-TTTGTCGTCTTTCCAGACGTTAGT-3; SEQ ID NO:48). The resulting PCR fragments were digested with BstNI (NEB) (2 hours, 37° C.) and analyzed by agarose gel electrophoresis.

Cell lines—JY (EBV-transformed B-lymphoblast), were maintained in RPMI-1640 supplemented with 10% fetal calf serum (FCS), 2 mM glutamine, Penicillin (100 units/ml) and Streptomycin (100 μg/ml) at 37° C. with 5% CO₂.

Expression and purification of soluble recombinant Fab antibodies—4 ml of miniprep DNA was transformed to 100 μl BL 21 E. coli competent cells and the bacteria was plated on 2YT/A/G agar plates and incubated at 37° C., over night. Inoculated plates were transferred into Superbroth supplemented with 12 ml/liter 40 gr/lit MgSO₄, 5 ml/liter 20% Glucose, and 100 μg/ml Ampicillin (5 plates full with colonies into each 1 liter of superbroth). The bacteria grew to OD₆₀₀ nm=0.8-1.0 and induced to express the recombinant Fab antibody by the addition of 1 mM IPTG for 3 hour at 30° C. The cells were centrifuged and the pellet was resuspended in 5 ml of a B-PER solution (Pierce) to release periplasmatic content. After 30 minutes of rotated incubation at room temperature (RT), the solution was centrifuged (15000 rpm, 15 minutes) and the supernatant was incubated with 0.5 ml of pre-washed TALON beads suspension (Clontech) for 45 minutes at RT. The solution was applied onto a Biorad disposable column, and after sedimentation the beads were washed three times with 10 ml of PBS/0.1% Tween-20 (pH 8.0). The bound Fabs were eluted using 0.5 ml of 100 mM Imidazole in PBS. The eluted Fabs were dialyzed twice against PBS (overnight, 4° C.) to remove residual imidazole. The homogeneity and purity of the purified Fabs was determined by analysis on non-reduced and reduced SDS-PAGE.

Production of fluorescent tetramerized Fabs—The genes encoding the light and heavy chain of Fab D12 were cloned separately into a T7-promotor pET-based expression vector. The light chain gene was engineered to contain the BirA recognition sequence for site-specific biotinylation at the COOH terminus (D12 light-BirA). These constructs were expressed separately in E. coli BL21 cells and upon induction with IPTG, intracellular inclusion bodies that contain large amounts of the recombinant protein accumulated. Inclusion bodies of both chains were purified, reduced, and subsequently refolded at a 1:1 ratio in a redox-shuffling buffer system containing 0.1 M Tris, 0.5 M Arginine, 0.09 mM Oxidized Glutathione (pH 8.0). Correctly folded Fab was then isolated and purified by anion exchange Qsepharose chromatography (Pharmacia). The Fab peak fractions were concentrated using Centricon 30 (Amicon) to 1 mg/ml, and the buffer was exchanged to Tris-HCl [10 mM (pH 8.0)]. Biotinylation was performed using the BirA enzyme (Avidity) as described previously. Excess biotin was removed from biotinylated Fabs using a G-25 desalting column. Phycoerythrin-labeled streptavidin (Jackson-Immunoresearch) was added at a molar ratio of 1:4 to produce fluorescent tetramers of the biotinylated Fab fragment.

ELISA with purified Fab antibodies—The binding specificity of individual soluble Fab fragments was determined by ELISA using biotinylated scMHC-peptide complexes. ELISA plates (Falcon) were coated overnight with BSA-biotin (1 μg/well). After having been washed, the plates were incubated (1 hour, RT) with streptavidin (1 μg/well), washed extensively and further incubated (1 hour, RT) with 0.5 μg of MHC/peptide complexes. Plates were blocked for 30 minutes at RT with PBS 2% BSA and subsequently were incubated for 1 hour at RT with various concentrations of soluble purified Fab, and after washing, with 1:1000 HRP-conjugated/anti-human antibody. Detection was performed using TMB reagent (Sigma). The HLA-A2-restricted peptides used for specificity studies of the purified Fab antibodies are: The influenza virus derived peptide M1(58-66) GILGFVFTL (SEQ ID NO:2), hTERT (540): ILAKFLHWL (SEQ ID NO:36) and hTERT (865): RLVDDFLLV (SEQ ID NO:37), MART derived peptide (26-35 modified): ELAGIGILTV (SEQ ID NO:38), EBV: GLCTLVAML (SEQ ID NO:39), CMV: NLVPMVATV (SEQ ID NO:40), HTLV-1 (TAX): LLFGYPVYV (SEQ ID NO:44), MUC1 (13-21): LLLTVLTVL (SEQ ID NO:45), gp100-G9-209-2M: IMDQVPFSV (SEQ ID NO:41), gp100-G9-280: YLEPGPVTA (SEQ ID NO:42), and gp100-154: KTWGQYWQV (SEQ ID NO:43).

Flow cytometry—The EBV-transformed B-lymphoblast JY cells or virus-infected cells as indicated were used to determine the reactivity of the recombinant T-cells receptor-like antibodies with cell surface-expressed HLA-A2/peptide complexes. About 10⁶ JY cells were washed with serum-free RPMI and incubated overnight at 37° C. in medium containing 100 μM of the peptide. The cells were incubated for 60 minutes at 4° C. with recombinant Fab antibodies (10-100 mg/ml) in 100 ml. After two washes the cells were incubated with FITC-labeled anti-human Fab or with PE-labeled anti-human Fab (Jackson) (for Fab Abs). After a final wash, the cells were resuspended in ice-cold PBS. The results were analyzed with the WinMDI program [Trotter, Hypertext Transfer Protocol://facs (dot) Scripps (dot) edu].

### Example 1

### Isolation of Antibodies which Specifically Bind the Complex of HLA-A 2 with the Influenza M1₅₈₋₆₆ Peptide

Experimental Results

Selection of TCR-like recombinant antibodies towards HLA-A2/M1₅₈₋₆₆—Recombinant peptide-HLA-A2 complexes that present the M1₅₈₋₆₆ influenza derived peptide (SEQ ID NO:2) were generated using a scMHC construct that was described previously (Denkberg, G., et al., 2000, European Journal of Immunology 30, 3522-3532). In this construct, the extracellular domains of HLA-A2 are connected into a single chain molecule with β2m using a 15-amino acid flexible linker (the β2m is upstream of the MHC heavy chain). The scMHC-peptide complexes were produced by in vitro refolding of inclusion bodies in the presence of the influenza-derived M1₅₈₋₆₆ peptide. The refolded scHLA-A2/M1 complexes were found to be very pure, homogenous, and monomeric by SDS-PAGE and size exclusion chromatography analyses (data not shown). Recombinant scMHC-peptide complexes generated by this strategy had been previously characterized in detail for their biochemical, biophysical, and biological properties, and were found to be correctly folded and functional [Denkberg, G., et al., 2000 (Supra); Denkberg, G., et al., 2001, Journal of Immunology 167, 270-276].

For selection of TCR-like Abs, a large Ab phage library was used, consisting of a repertoire of 3.7×10¹⁰ independent human recombinant Fab clones (de Haard, H. J. et al., 1999). The selection strategy included depletion of the library of streptavidin binders and subsequently panning the library in solution using soluble recombinant scHLA-A2-peptide complexes containing the M1₅₈₋₆₆ peptide. A 200-fold enrichment in phage titer was observed after three rounds of panning. The specificity of the selected phage Abs was determined by a differential ELISA analysis on streptavidin-coated wells incubated with biotinylated scMHC HLA-A2 complexes containing either the M1₅₈₋₆₆ peptide or control complexes containing other HLA-A2-restricted peptides (FIG. 1). Phage clones analyzed after the third round of selection exhibited two types of binding patterns toward the scHLA-A2-peptide complex: one class of Abs consisted of pan-MHC binders that cannot differentiate between the various MHC-peptide complexes; the second type consisted of Abs that bound the MHC/peptide complex in a peptide-specific manner. The ELISA screen revealed that 70 of 90 randomly selected clones screened (77%) from the third round of panning appeared to be fully peptide dependent and specific for the peptide/MHC used in the selection (i.e., the scHLA-A2/M1 complex).

A representative analysis of eight (A2, D12, D1, E6, F8, F11, B12 and H8) TCR-like Fab clones that reacted only with the scHLA-A2/M1 (SEQ ID NO:2) complex and not with control scHLA-A2/peptide complexes in which the peptide is hTERT-540 (SEQ ID NO:36), hTERT-865 (SEQ ID NO:37) or Mart (SEQ ID NO:38) is shown in FIG. 2.

Characterization of recombinant soluble Fab Antibodies with TCR-Like specificity—The Fab clones were produced in a soluble form in E. coli TG1 or BL21 cells and purified by immobilized metal ion affinity chromatography (IMAC). Yields were 2-4 mg of pure material from 1 liter of bacterial culture. SDS-PAGE analysis revealed a homogenous and pure population of Fabs with the expected molecular size.

The binding specificity of the purified Fab fragments was determined by ELISAs on biotinylated MHC-peptide complexes immobilized to wells through BSA-biotin-streptavidin. The correct folding of the bound complexes and their stability during the binding assays were determined by their ability to react with the conformational specific monoclonal antibody W6/32, which binds HLA complexes only when folded correctly and when it contains peptide (data not shown). When the soluble purified Fabs were analyzed by ELISA, a very specific recognition pattern was revealed (FIG. 3). The Fab clones (e.g., D12, D1, F8 and G8), selected to bind the HLA-A2/M1₅₈₋₆₆ complex, bound only to the specific complex but not to other control HLA-A2/peptide complexes displaying various tumor or viral-derived T-cell epitopes (peptides) (FIG. 3). In control experiments, the Fab clones did not bind the antigenic peptide (M158-66) when not in complex with the specific MHC molecule (i.e., HLA-A2) that formed the MHC-peptide complex against which the antibody was selected (data not shown). One of the Fab clones (clone D12) which was selected against the HLA-A2/M1 complex and exhibited the most specific peptide-dependent and TCR-like binding pattern as analyzed by the phage ELISAs was used for further analysis.

FIGS. 7A-D depict sequences of the D12 antibody which specifically binds the HLA-A2/M1 complex.

These results demonstrate that the peptide-specific, MHC-restricted Fabs exhibited binding characteristic and fine specificity of a TCR-like molecule.

### Example 2

### Characterization of the Binding of the D12 Antibody to Cells Presenting the M1-Derived Epitope

Experimental Results

Binding of D12 Fab antibody to APCs displaying the M1-derived epitope—To demonstrate that the isolated soluble Fab antibodies can bind the specific MHC-peptide complex not only in its recombinant soluble form but also in the native form as expressed on the cell surface, the TAP⁺ Epstein-Barr virus (EBV)-transformed B lymphoblast JY cells, which express HLA-A2 were incubated with the influenza M1-derived peptide or control peptides. These cells express TAP (transporter associated with antigen processing), and consequently display the exogenous peptide by peptide exchange. Using this strategy, a mixture of exogenously and endogenously derived peptides presented on HLA-A2 that are displayed on the cell surface was obtained. In testing the HLA-A2/M1₅₈₋₆₆-specific antibodies, intensive staining of JY cells loaded with the specific M1₅₈₋₆₆ derived peptide but not with the other control peptides was observed (FIGS. 4A-L and FIG. 5).

These results demonstrate the ability of the TCR-like antibodies to detect the specific MHC/M1₅₈₋₆₆ complex on the surface cells.

### Example 3

### Generation of Fab-Tetramers which Bind Complexes of HLA-A2/M1 with Increased Avidity

The density of a particular peptide-HLA complex on cells is expected to be low compared to peptide-pulsed APCs. In order to improve the avidity and sensitivity of the isolated D12 Fab to cells presenting the complex (HLA-A2/M1), the present inventors have generated Fab-tetramers, which are directly tagged with a fluorescent probe. This approach was used previously to increase the binding avidity of peptide-MHC complexes to the TCR or to increase the sensitivity of recombinant Ab molecules (Cloutier, S. M. et al. 2000, Molecular Immunology 37, 1067-1077). Another advantage of using fluorescently labeled tetramers is that only a single staining step is required, whereas monomeric unlabeled Fabs require a fluorescently labeled secondary Ab.

### Experimental Results

Increasing the avidity of TCR-like Fab D12—To generate Fab tetramers the BirA peptide sequence was introduced at the C-terminus of the light chain for site-specific biotinylation. Recombinant D12 Fab was refolded by in vitro refolding as described under “General Materials and Experimental Methods” and was subjected to in vitro biotinylation by the E. coli BirA enzyme as described previously [Cloutier, S. M. et al. 2000 (Supra)]. The D12 Fab tetramer, which was generated with fluorescently labeled streptavidin, was used to measure the expression of HLA-A2/M1₅₈₋₆₆ complexes on the surface of peptide pulsed APCs. As shown in FIGS. 6A-B, the fluorescence intensity measured on peptide pulsed JY cells upon the binding of the D12 Fab tetramer by flow cytometry (FIG. 6B) was significantly improved compared with the reactivity of the Fab monomer (FIG. 6A).

These results further demonstrate the ability of these high affinity TCR-like Abs to detect the specific MHC-peptide complex on the surface of APCs.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Additional References are Cited in Text

1. de Haard, H. J. et al. A large non-immunized human Fab fragment phage library that permits rapid isolation and kinetic analysis of high affinity antibodies. Journal of Biological Chemistry 274, 18218-18230 (1999).
2. Brinkmann, U., Pai, L. H., Fitzgerald, D. J., Willingham, M. & Pastan, I. B3(Fv)-Pe38Kdel, A Single-Chain Immunotoxin That Causes Complete Regression of A Human Carcinoma in Mice. Proceedings of the National Academy of Sciences of the United States of America 88, 8616-8620 (1991).
3. Denkberg, G., Cohen, C. J., Segal, D., Kirkin, A. F. & Reiter, Y. Recombinant human single-chain MHC-peptide complexes made from E-coli by in vitro refolding: functional single-chain MHC-peptide complexes and tetramers with tumor associated antigens. European Journal of Immunology 30, 3522-3532 (2000).
4. Denkberg, G., Cohen, C. J. & Reiter, Y. Critical role for CD8 in binding of MHC tetramers to TCR: CD8 antibodies block specific binding of human tumor-specific MHC-Peptide tetramers to TCR. Journal of Immunology 167, 270-276 (2001).
5. Cloutier, S. M. et al. Streptabody, a high avidity molecule made by tetramerization of in vivo biotinylated, phage display-selected scFv fragments on streptavidin. Molecular Immunology 37, 1067-1077 (2000).
6. Pastan, I. Targeted therapy of cancer with recombinant immunotoxins. Biochimica et Biophysica Acta-Reviews on Cancer 1333, C1-C6 (1997).
7. Dalod, M. et al. Weak anti-HIV CD8(+) T-cell effector activity in HIV primary infection. J. Clin. Invest 104, 1431-1439 (1999).
8. Goulder, P. J. et al. Substantial differences in specificity of HIV-specific cytotoxic T cells in acute and chronic HIV infection. J. Exp. Med. 193, 181-194 (2001).

1. An isolated antibody comprising an antigen recognition domain capable of binding an MHC molecule being complexed with an influenza peptide derived from an influenza polypeptide selected from the group consisting of SEQ ID NOs: 19, 20, 21, 22, 23, 24, 25, 26, 27, 28, 29, 30, 31, 32, 33, 34 and 35, wherein the antibody does not bind said MHC molecule in an absence of said complexed peptide, and wherein the antibody does not bind said peptide in an absence of said MHC molecule.

2. The isolated antibody of claim 1, wherein said antigen recognition domain comprises complementarity determining region (CDR) amino acid sequences as set forth in SEQ ID NOs:3-8.

3. A molecule comprising the antibody of claim 1 conjugated to a therapeutic moiety.

4. A molecule comprising the antibody of claim 1 conjugated to a detectable moiety.

5. The antibody of claim 1, being multivalent.

6. The antibody of claim 5, being of an IgG class.

7. A multivalent composition comprising the isolated antibody of claim 1.

8. An isolated polynucleotide comprising a nucleic acid sequence encoding the antibody of claim 1.

9. The isolated polynucleotide of claim 8, wherein said nucleic acid sequence comprises SEQ ID NOs:9-14.

10. A nucleic acid construct comprising the isolated polynucleotide of claim 8 and a promoter for directing expression of said nucleic acid sequence in a host cell.

11. A pharmaceutical composition comprising as an active ingredient the antibody of claim 1.

12. A method of detecting a cell expressing an influenza antigen, comprising contacting the cell with the antibody of claim 1, under conditions which allow immunocomplex formation, wherein a presence or a level above a predetermined threshold of said immunocomplex is indicative of influenza expression in the cell.

13. A method of diagnosing an influenza infection in a subject in need thereof, comprising contacting a biological sample of the subject with the antibody of claim 1, under conditions which allow immunocomplex formation, wherein a presence or a level above a pre-determined threshold of said immunocomplex in the biological sample is indicative of the influenza infected-cells in the subject, thereby diagnosing influenza infection in the subject.

14. A method of treating an influenza infection, comprising administering to a subject in need thereof a therapeutically effective amount of the antibody of claim 1, thereby treating the influenza infection.

15. The method of claim 14, wherein said antibody, is capable of killing influenza-infected cells in the subject.

16. (canceled)

17. (canceled)

18. A method of treating an influenza infection, comprising administering to a subject in need thereof a therapeutically effective amount of the nucleic acid construct of claim 10, thereby treating the influenza infection.
